(19) United States
(12) Patent Application Publication
(10) Pub. No.: US 2026/0151413 A1
(43) Pub. Date: Jun. 4, 2026

(54) ANTICANCER DRUG CONJUGATES

(71) Applicant: Ariel Scientific Innovations Ltd., Ariel (IL)

(72) Inventor: Gary GELLERMAN, Rishon-LeZion (IL)

(73) Assignee: Ariel Scientific Innovations Ltd., Ariel (IL)

(21) Appl. No.: 19/123,789

(22) PCT Filed: Oct. 18, 2023
(86) PCT No.: PCT/IL2023/051087

§ 371 Date: Apr. 24, 2025

Related U.S. Application Data
(60) Provisional application No. 63/418,666, filed on Oct. 24, 2022.

(51) Int. Cl. A61K 31/704; A61P 35/00; C07H 15/24

(52) U.S. Cl. CPC A61K 31/704; A61P 35/00; C07H 15/24

(57) ABSTRACT

Anticancer conjugates combining a DNA methylation agent and an anticancer drug are provided herein, as well as uses of the same in cancer treatment.

### RELATED APPLICATION

This application claims the benefit of priority of U.S. Provisional Patent Application No. 63/418,666 filed 24 Oct. 2022, the content of which is incorporated herein by reference in its entirety.

### FIELD AND BACKGROUND OF THE INVENTION

The present invention, in some embodiments thereof, relates to pharmaceutical agents, and more particularly, but not exclusively, to dual-action conjugates useful as anticancer agents.

Enhancing the therapeutic efficacy of drugs while minimizing undesirable side effects is a perpetual challenge in the realm of drug development. Traditional drug design often revolves around refining single agents, but in recent years, an innovative approach has gained traction. This approach involves the creation of structural hybrids, commonly referred to as drug conjugates or chimeras, which amalgamate the pharmacophores of multiple drugs into a single molecular entity. This novel strategy allows for the simultaneous targeting of multiple cellular sites or molecular targets, resulting in the emergence of dual-action pharmaceutical agents.

The pursuit of dual-action pharmaceutical agents marks a transformative shift in drug design, promising a new era of therapeutic interventions. By concurrently engaging with distinct cellular pathways or multiple molecular targets, these agents offer the potential for synergistic therapeutic effects while mitigating adverse reactions. This multifaceted approach has garnered considerable attention across a spectrum of therapeutic domains, with a pronounced focus on anticancer applications.

In recent years, researchers have made significant strides in the design, synthesis, and evaluation of novel dual-action pharmaceutical agents. Notably, various drug conjugates have emerged as exemplars of this innovative approach. For instance, conjugates comprising two distinct anticancer drugs have demonstrated remarkable potential. These conjugates, designed to simultaneously target multiple aspects of tumorigenesis, present a compelling strategy in the fight against cancer.

One notable example is the gemcitabine-cisplatin hybrid, which combines two well-established anticancer agents, gemcitabine and cisplatin, into a single molecular entity. This approach has shown promising results by concurrently targeting multiple cellular pathways involved in tumor growth and proliferation. Gemcitabine-cisplatin conjugate is currently in clinical development for the treatment of a variety of cancers, including pancreatic cancer, lung cancer, and bladder cancer.

Duocarmycin-paclitaxel conjugate (DSP) is a dual drug conjugate that combines the cytotoxic drug duocarmycin with the microtubule-targeting agent paclitaxel. DSP is currently in clinical development for the treatment of advanced solid tumors.

Doxorubicin-paclitaxel conjugate (DPX) is a dual drug conjugate that combines the cytotoxic drugs doxorubicin and paclitaxel. Doxorubicin-paclitaxel conjugate is currently in clinical development for the treatment of a variety of cancers, including breast cancer, lung cancer, and pancreatic cancer.

Camptothecin-topoisomerase I inhibitor conjugate (CTI) is a dual drug conjugate that combines the cytotoxic drug camptothecin with a topoisomerase I inhibitor. CTI is currently in clinical development for the treatment of a variety of cancers, including lung cancer, ovarian cancer, and colorectal cancer.

Vincristine-cisplatin conjugate is a dual drug conjugate that combines the cytotoxic drugs vincristine and cisplatin. Vincristine-cisplatin conjugate is currently in clinical development for the treatment of a variety of cancers, including lung cancer, breast cancer, and ovarian cancer.

A monomethyl triazene moiety is a chemical group consisting of a triazene ring with a single methyl group attached. Monomethyl triazenes are important agents in the field of cancer chemotherapy. Several monomethyl triazene derivatives have been developed as anticancer drugs, including temozolomide (Temodar) and dacarbazine (DTIC). These drugs work by alkylating DNA, which can damage cancer cells and lead to their death. Temozolomide is a first-line treatment for glioblastoma, the most common type of primary brain cancer. It is also used to treat other types of brain cancer, such as anaplastic astrocytoma and medulloblastoma. Dacarbazine is used to treat a variety of cancers, including malignant melanoma, Hodgkin lymphoma, and non-Hodgkin lymphoma.

Doxorubicin (also known as Adriamycin) is a chemotherapy drug used to treat a variety of cancers, including breast cancer, bladder cancer, Kaposi's sarcoma, lymphoma, and leukemia. It is given by injection into a vein. Doxorubicin is a type of anthracycline antibiotic, which are a class of chemotherapy drugs that work by damaging DNA and preventing cancer cells from dividing. Doxorubicin is a very effective chemotherapy drug, but it can also cause serious side effects, such as heart damage, hair loss, and nausea and vomiting.

WO 2017/216791 provides conjugates that combines two residues of structurally and/or mechanistically different anticancer bioactive agents, coupled to one another by a biocleavable linking moiety, as well as methods of treating cancer using the same and pharmaceutical compositions comprising the same.

Amonafide is a small molecule that intercalates into DNA and inhibits topoisomerase II activity, which is essential for DNA replication and repair, whereas inhibition of topoisomerase II can lead to DNA double-strand breaks, which can cause cell death. Monomethyl triazenes are a class of alkylating agents that can damage DNA by attaching methyl groups to DNA bases, which leads to DNA mutations and cell death. Amonafidazene, developed by the present inventor [Walunj, D. et al., “Targeted methylation facilitates DNA double strand breaks and enhances cancer suppression: A DNA intercalating/methylating dual-action chimera Amonafidazene”, 2021, European Journal of Medicinal Chemistry, 225, 113811], is a chimeric molecule that combines the DNA intercalating/methylating properties of Amonafide with the DNA alkylating properties of monomethyl triazene. It is a potential anticancer drug that is currently in preclinical development.

Yet, there is still a need for effective pharmaceuticals, including, but not limited to anti-cancer drugs in the form of dual activity conjugates.

### SUMMARY OF THE INVENTION

Modifying existing drugs to enhance their activity and reduce side effects is a major direction of drug design efforts. One way to address this challenge is to merge their active parts into structural entities referred to herein as “conjugates”. Provided herein is a class of dual-action conjugates (chimeras) useful in treating cancer, which consist of known drugs conjugated to methyl carbamate protected DNA methylating monomethyl triazene moiety, referred to herein as Azene, by a simple nucleophilic substitution.

The in vitro screening of some exemplary conjugates, according to some embodiments of the present invention, on a battery of cancer cell lines, revealed several highly potent Azene conjugates of Doxorubizen (a derivative of the drug doxorubicin that is a topoisomerase II inhibitor). It was found that in all examined cancer cell lines, significantly enhanced nanomolar cytotoxicity, apoptosis and mitochondria depolarization over an equimolar concentration of all tested conjugates and their parent drugs. It is assumed that the mechanism of action of Doxorubizen is associated with the inhibition of DNA repair in the proximity to the double strand breaks (DSB) by Guanine methylation, enhancing cancer cell apoptosis. In negative breast cancer (TNBC) xenograft model, Doxorubizen was profoundly superior to a parent Dox in reduction of tumor growth, maintenance of body weight and extension of overall survival.

Thus, according to an aspect of some embodiments of the present invention, there is provided a compound that is also referred to herein as “conjugate” or “chimera”, which is defined and characterized by general Formula I:

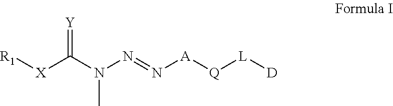

wherein:
D is a residue of a bioactive agent;
L is a linking moiety that is not carbamate;
A is an aromatic or a heteroaromatic ring;
Q is selected from the group consisting of (CH2)1-10, C═O, C═NH, C═N-alkyl, C═N-aryl and C═S;
X and Y are each independently selected from the group consisting of O, S, N, NH, alkylene C₁₋₅, sulfone, and phosphonate; and
R₁ is selected from the group consisting of hydrogen, alkyl, alkoxyalkyl, alkoxy, aryl, heteroaryl, alkylaryl, o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, a tertiary alkyl amine, a quaternary ammonium choline, L-valine-L-citrulline-p-aminobenzyl alcohol (Val-Cit-PABA), L-valine-L-alanine-p-aminobenzyl alcohol (Val-Ala-PABA), or a protecting group.

In some embodiments, variable D is an anticancer drug selected from the group consisting of Aldoxorubicin, Amonafide, Amrubicin, and Monomethyl auristatin E (MMAE), Annamycin, Berubicin, Camsirubicin, Carubicin, Combretastatin, Daunomustine, Daunorubicin (Daunomycin), Daunorubicin (Daunomycin), Doxorubicin, Doxorubicin, Doxorubicin, Doxorubicin (Adriamycin), Epirubicin (Ellence), Etoposide, 5-Fluorouracil, Idarubicin (Idamycin), Lenalidomide, Losoxantrone, 6-Mercaptopurine, Mitoxantrone, Mitoxantrone, Pirarubicin, Pixantrone, Riluzole, Sabarubicin, SN-38, and Zorubicin.

In some embodiments, variable L is selected from the group consisting of amine, ether, thioether, amide, carbonate, lactone, lactam, carboxylate, ester, boroalkyl, boronate, sulphone, sulphate, phosphonate, phosphine, phosphite, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thiocarbamate, thioacetal, thioketal, phosphate ester, and the like.

In some embodiments, variable L is amine, ether, thiocarbamate, carbonate, or thioether. In some embodiments, variable L is not carbamate.

In some embodiments, variable A is a substituted or unsubstituted aromatic or heteroaromatic ring selected from the group consisting of phenyl, naphthyl, anthryl, phenanthryl, biphenyl, furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, thiazole, bicyclo[4.4.0], bicyclo[4.3.0], indolizine, indole, isoindole, indazole, benzimidazole, benzthiazole, purine, quinoline, isoquinoline, cinnoline, phthalazine, quinazoline, quinoxaline, 1,8-naphthyridine, and pteridine.

In some embodiments, variable A is phenyl.

In some embodiments, variables X and Y are each independently O.

In some embodiments, variable Q is —CH₂—.

In some embodiments, the compound provided herein is selected independently from the group consisting of Chimera 1, Chimera 2, Chimera 3, Chimera 4, Chimera 5, Chimera 6, Chimera 7, Chimera 8, Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 23, Chimera 24, Chimera 25, Chimera 26, Chimera 27, Chimera 28, Chimera 29, and Chimera 30.

According to an aspect of some embodiments o the, there is provided a compound, defined and characterized by general Formula II:

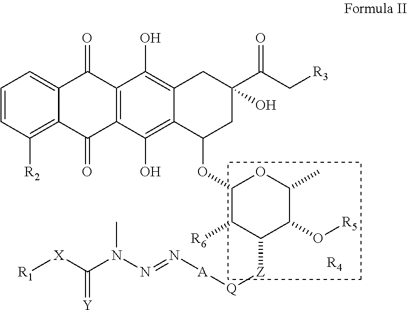

wherein:
R₁ is selected from the group consisting of hydrogen, alkyl, alkoxyalkyl, alkoxy, aryl, heteroaryl, alkylaryl, o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, a tertiary alkyl amine, a quaternary ammonium choline, L-valine-L-citrulline-p-aminobenzyl alcohol (Val-Cit-PABA), L-valine-L-alanine-p-aminobenzyl alcohol (Val-Ala-PABA), or a protecting group;
each of R₂ and R₃ is independently selected from the group consisting of hydrogen, hydroxy, alkyl, alkoxyalkyl, alkoxy, aryl, heteroaryl, alkylaryl, ortho nitrobenzyl, benzhydryl, trityl, alkylsilyl, or a protecting group; R₄ is selected from the group consisting of the (2R,4S,5S,6S), (2R,4S,5R,6S), (2R,4S,5S,6S) and (2R,4S,5S,6S) stereoisomer of 4-(kl-azaneyl)-2-methyl-6-(λ²-oxidaneyl)tetrahydro-2H-pyran-3-ol;
R₅ is selected from the group consisting of hydrogen, alkyl, aryl, heteroaryl and benzyl;
R₆ is selected from the group consisting of hydrogen, F, Cl, Br and I;
Z is selected from the group consisting of O, S, NH and N-alkyl;
Q is selected from the group consisting of (CH₂)₁₋₁₀, C═O, C═NH, C═N-alkyl, C═N-aryl and C═S;
A is an aromatic or a heteroaromatic ring; and

In some embodiments, variable Q is —CH₂—.

X and Y are each independently selected from the group consisting of O, S, N, NH, alkylene (C₁₋₅), sulfone, and phosphonate.

In some embodiments, variables R₅ and R₆ is each independently hydrogen.

In some embodiments, variable Z is NH.

In some embodiments, variable Q is CH₂.

In some embodiments, variable A is phenyl.

In some embodiments, variables X and Y are each O.

In some embodiments, the compound is selected independently from the group consisting of Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 24, Chimera 25, Chimera 26, Chimera 27, Chimera 28, Chimera 29, and Chimera 30.

According to another aspect of some embodiments of the present invention, there is provided a compound selected independently from the group consisting of Chimera 1, Chimera 2, Chimera 3, Chimera 4, Chimera 5, Chimera 6, Chimera 7, Chimera 8, Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 23, Chimera 24, Chimera 25, Chimera 26, Chimera 27, Chimera 28, Chimera 29, and Chimera 30.

According to another aspect of some embodiments of the present invention, there is provided a compound as provided herein, and/or any pharmaceutically acceptable salt, a prodrug, an ester, a solvate, and/or hydrate thereof, for use in the treatment of cancer.

According to another aspect of some embodiments of the present invention, there is provided a pharmaceutical composition that includes a therapeutically effective amount of the compound provided herein, and/or any pharmaceutically acceptable salt, a prodrug, an ester, a solvate, and/or hydrate thereof, and a pharmaceutically acceptable carrier, diluent or vehicle.

According to another aspect of some embodiments of the present invention, there is provided a use of the compound provided herein and/or any pharmaceutically acceptable salt, a prodrug, an ester, a solvate, and/or hydrate thereof for the manufacture of a medicament for the treatment of cancer.

According to another aspect of some embodiments of the present invention, there is provided a compound as provided herein and/or any pharmaceutically acceptable salt, a prodrug, an ester, a solvate, and/or hydrate thereof, or a composition provided herein, for use in treating cancer.

According to another aspect of some embodiments of the present invention, there is provided a method for treating cancer, which is effected by administering to a subject in need thereof a pharmaceutically effective amount of the compound provided herein and/or any pharmaceutically acceptable salt, a prodrug, an ester, a solvate, and/or hydrate thereof, or a composition as provided herein.

### DESCRIPTION OF EMBODIMENTS OF THE INVENTION

The present invention, in some embodiments thereof, relates to pharmaceutical agents, and more particularly, but not exclusively, to dual-action conjugates useful as anticancer agents.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

To provide new conjugated drugs entities, the present inventor contemplated the conjugation of anticancer drugs with a protected form of a DNA methylating agent. monomethyl triazene moiety, also referred to herein as “azene”. Unlike in the case of Amonafidazene [Walunj, D. et al., European J Med Chem, 225, 113811], the present invention is drawn to conjugates exhibiting a different bio-triggering mechanism, which is afforded by brominating the azene moiety (CBr4, PPh3) to afford azene bromide alkylated directly (K2CO3, DMF) to a bio-triggering group (hydroxyl, amine, thiol etc.) of the corresponding drug. Such an approach yields dual-function conjugates that are activated regio-selectively and solely through the bio-deprotection of the methyl carbamate (MeCO2N—) protection group of mono-methyl triazene tether. This mechanism leads to the cascade-like degradation, initially releasing the DNA methylating methyl carbocation and consequently the drug itself. Amonafidazene, however, has two carbamate groups lacking such regio-specific activation enabling a non-specific disconnection of entire Azene tether before releasing Me+ in proximity to Amonafide DNA intercalation site and therefore neutralize augmentation of the efficacy of DNA intercalating Amonafide. Such a proximity of the Azene to the parent drug at the target is crucial for increasing efficacy. In the conjugates provided herein, according to embodiments of the present invention, the Azene moiety can augment the efficacy of DNA intercalating Amonafide because of releasing Me+ from Azene close to the parent DNA intercalator at the target. Another merit of using Azene bromide in direct alkylation was exemplified on amino containing drugs, such as amonafide itself and anthracyclines, in particular doxorubicin. Upon alkylation, it forms the secondary amine on the parent drug that can be quaternized with protic acid (e.g., HCl, HOAc) to the corresponding salt or linked to solubilizer increasing the solubility of the afforded chimeras what is not possible in case of Amonafidazene. It is noted herein that the conjugate referred to herein as Amonafidazene, which exhibits a carbamate linking moiety between the Azene and the Amonafide is excluded from the scope of the claims defining the present invention.

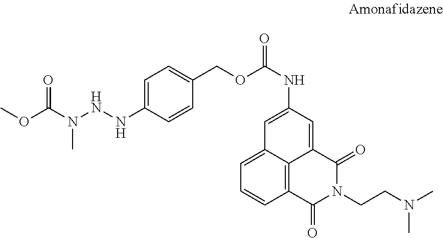

While conceiving the present invention, the present inventor envisioned a molecular chimera or conjugate that exhibits two bioactive moieties, one is a known drug (a bioactive agent), and the other is a moiety of a methylating agent methyl-(E)-1-methyl-3-(p-tolyl)triaz-2-ene-1-carboxylate (Azene, MMTA; MTTC) and derivatives thereof.

According to some embodiments of the present invention the general formula of the radical of the “Azene” moiety is presented below:

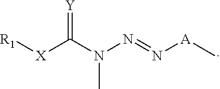

wherein A is an aromatic or a heteroaromatic ring, including rings of aromatic amino acids (naturally occurring; synthetic; derivatives thereof), which allow the moiety to be tethered into peptides;
X and Y are each independently selected from the group consisting of O, S, N, NH, alkylene (C₁₋₅), sulfone, and phosphonate (one form of a triazene protecting group or another); and
R₁ is selected from the group consisting of hydrogen, alkyl (e.g., methyl, ethyl, propyl, etc.), alkoxyalkyl (e.g., -alkyl-O-alkyl, methoyalkyl, methoxyethyl, 1-methoxypropyl, 1-ethoxy-2-methoxyethyl; 1-(2-methoxyethoxy)propyl etc.), alkoxy (e.g., —O-alkyl, methoxy, ethoxy, etc.), aryl/heteroaryl (e.g., phenyl, pyridine, etc.), alkylaryl (e.g., benzyl), o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, tertiary alkyl amines like Me₂NCH₂CH₂OH, quaternary ammonium choline (e.g., Me₃N⁺CH₂CH₂OH), Val-Cit-PABA, Val-Ala-PABA, or a protecting group.

When A is a ring of aromatic amino acid, exemplary Azene moieties, which can be tethered into peptides, include, without limitation, the following examples:

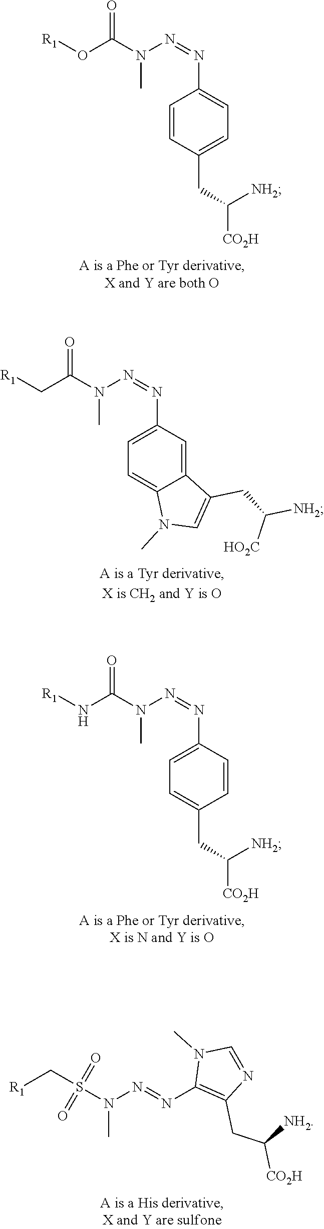

While reducing the present invention to practice, the present inventor has demonstrated that the conjugates provided herein, which combine DNA intercalating and DNA methylating activities in one molecular entity (chimera or conjugate) are superior to the standalone drugs in numerous hematopoietic and solid cancer models. This superior anticancer activity, characteristic to the conjugates according to some embodiments of the present invention, is compared to that of the activity of each of the conjugate moieties active alone.

The present inventor demonstrated the above approach on: (1) Topo2 inhibitors amonafide, doxorubicin and etoposide; (2) microtubule inhibitor combretastin; (3) Topo1 inhibitor SN-38; (4) antimetabolites 5-fluorouracil, 6-mercaptopurine; (5) anti-angiogenic lenalidomide; (6) neuroprotective drug with potential as a novel anti-cancer agent riluzole. These drugs were reacted with Azene bromide to create small library of chimeric conjugates which were screened against a battery of cell lines including solid and hematological malignancies. One chimera, referred to herein as “Doxorubizen”, a hybrid of Doxorubicin (Dox; also known as Adriamycin) and Azene, was found potent in all tested cell lines at nanomolar range significantly enhanced cytotoxicity, apoptosis and mitochondria depolarization over an equimolar concentration of the parent drug.

### Anticancer Conjugates:

Thus, according to some embodiments of the present invention, there is provided a compound, characterized by general Formula I:

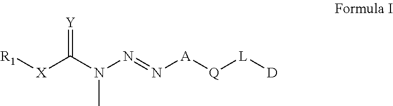

wherein:
D is a residue (a major moiety) of a bioactive agent (i.e., a drug; e.g., an anticancer drug; DNA intercalating agent);
L is a linking moiety;
A is an aromatic or heteroaromatic ring;
Q is selected from the group consisting of (CH2)1-10, C═O, C═NH, C═N-alkyl, C═N-aryl and C═S;
X and Y are each independently O, S, N, NH, alkylene C₁₋₅, sulfones, phosphonate, and any combination that forms a triazene protecting groups as these are known in the art; and
R₁ is selected from the group consisting of hydrogen, alkyl, alkoxyalkyl, alkoxy, aryl, heteroaryl, alkylaryl, ortho nitrobenzyl, benzhydryl, trityl, alkylsilyl, perillyl alcohol, and the likes, or a protecting group.

Alternatively, R₁ is selected from the group consisting of hydrogen, alkyl (e.g., methyl, ethyl, propyl, etc.), alkoxyalkyl (e.g., -alkyl-O-alkyl, methoyalkyl, methoxyethyl, 1-methoxypropyl, 1-ethoxy-2-methoxyethyl; 1-(2-methoxyethoxy)propyl etc.), alkoxy (e.g., —O— alkyl, methoxy, ethoxy, etc.), aryl/heteroaryl (e.g., phenyl, pyridine, etc.), alkylaryl (e.g., benzyl), o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, tertiary alkyl amines like Me₂NCH₂CH₂OH, quaternary ammonium choline (e.g., Me₃N⁺CH₂CH₂OH), L-valine-L-citrulline-p-aminobenzyl alcohol (Val-Cit-PABA), L-valine-L-alanine-p-aminobenzyl alcohol (Val-Ala-PABA), or a protecting group.

Alternatively, X and Y are each independently O, S, N or NH.

In some embodiments, A is phenyl or any substituted or unsubstituted aryl, heteroaryl, and any simple or substituted aromatic ring, such as, without limitation, furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, benzene, naphthalene, anthracene, pyridine, quinoline, isoquinoline, pyrazine, quinoxaline, acridine, pyrimidine, quinazoline, pyridazine, cinnoline, phthalazine, 1,3,5-triazine, 1,2,3-triazine, 1,2.4-triazine (s-triazine), and the likes.

Some exemplary non-limiting alternatives to the phenyl ring in the Azene moiety are presented hereinbelow in the context of the conjugate compound defined by Formula I:

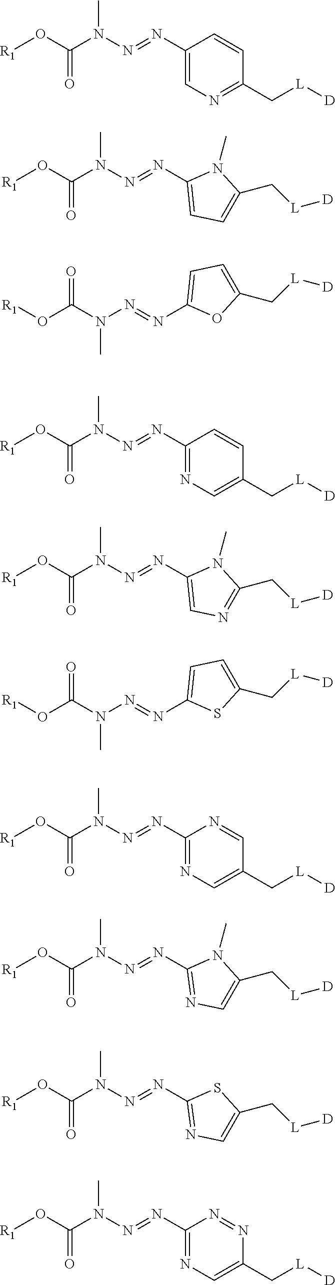

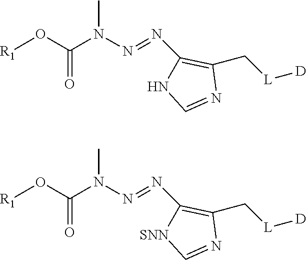

In some embodiments, the “methylating agent” functionality in the conjugate provided herein takes the form of the Azene moiety, as shown in Formula I(1):

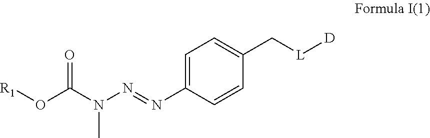

wherein A of Formula I is phenyl, and X and Y of Formula I are both oxygen atoms defining a carbamate-type triazene protecting group.

In some embodiments, and as demonstrated in the Example section below, D is an anticancer drug selected from the group consisting of Doxorubicin (Adriamycin), Daunorubicin (Daunomycin), Epirubicin (Ellence), Idarubicin (Idamycin), Annamycin, Camsirubicin, Berubicin, Daunorubicin (Daunomycin), Daunomustine, SN-38, Lenalidomide, Etoposide, Combretastatin, 5-Fluorouracil, 6-Mercaptopurine, Riluzole, Amonafide, Lenalidomide, Etoposide, Combretastatin, 5-Fluorouracil, 6-Mercaptopurine, Riluzole, Amonafide, Zorubicin, Carubicin, Losoxantrone, Pixantrone, Mitoxantrone, Mitoxantrone, Doxorubicin, Doxorubicin, Doxorubicin, Pirarubicin, Sabarubicin, Amrubicin, Aldoxorubicin, and Monomethyl auristatin E (MMAE).

It is noted that the bioactive agent in the conjugate depicted in Formula I, namely D, can be any molecule (e.g., bioactive agent) that exhibits a functional group suitable for conjugation with the aryl/heteroaryl, or the tolyl moiety in Azene.

### Linking Moiety:

The atoms that tether the Azene moiety to the drug moiety is referred to herein and in Formula I as a linking moiety. As discussed hereinabove, the nature of the linking moiety has an effect on the bioactivity and effectivity of the conjugate, hence the present inventio provides conjugates that exhibit improved activity, which is related to the selection of the linking moiety, compared to amonafidazene [Walunj, D. et al., Eur. J. Med. Chem., 2021, 225, 113811].

Variable “L” in Formula I represents a linking moiety, linking between the moiety of the methylating agent and the moiety of the bioactive agent (drug; intercalating agent). The linking moiety is a result of the conjugation reaction between the two parts of the conjugate, and can be a bond (a pair of electrons forming a covalent bond), an atom—typically a heteroatom (N, O, S, and the like), or a group of atoms.

As used herein, the words “link”, “linked”, “linkage” “linker”, “bound” or “attached”, are used interchangeably herein and refer to the presence of at least one covalent bond between species, unless specifically noted otherwise. As used herein, the term “moiety” describes portion of a molecule, and typically a major portion thereof, or a group of atoms pertaining to a specific function.

As used herein, the term “linking moiety” describes a chemical moiety (a group of atoms or a covalent bond) that links two chemical moieties via one or more covalent bonds. A linking moiety may include atoms that form a part of one or both of the chemical moieties it links, and/or include atoms that do not form a part of one or both of the chemical moieties it links. For example, a peptide bond (amide) linking moiety that links two amino acids includes at least a nitrogen atom and a hydrogen atom from one amino acid and at least a carboxyl of the other amino acid. In general, the linking moiety can be formed during a chemical reaction, such that by reacting two or more reactive groups, the linking moiety is formed as a new chemical entity which can comprise a bond (between two atoms), or one or more bonded atoms. Alternatively, the linking moiety can be an independent chemical moiety comprising two or more reactive groups to which the reactive groups of other compounds can be attached, either directly or indirectly, as is detailed hereinunder.

As discussed hereinabove, the Azene moiety was previously employed by the present inventor to turn the anticancer drug Amonafide into the conjugate Amonafidazene by linkage of Azene to Amonafide through a carbamate linking moiety [Walunj, D. et al., Eur. J. Med. Chem., 2021, 225, 113811].

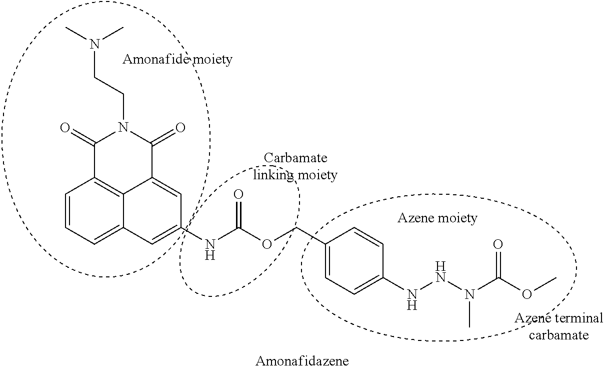 [SMILES: COC(=O)N(C)NNc1ccc(COC(=O)Nc2cc3c4c(cccc4c2)C(=O)N(CCN(C)C)C3=O)cc1]

As can be seen in the above scheme, Amonafidazene contains two biodegradable carbamate groups: one is the Azene terminal, connected to monomethyltriazene, and the second is the linking moiety between Azene and Amonafide. In contrast, and according to some embodiments of the present invention, the two major moieties of the conjugates are linked by an Azene linking position wherein the hydroxy group is replaced by a halo (Br), allowing direct alkylation of the amine, thereby forming a secondary amine that is more biostable (less biodegradable), at least by comparison with the carbamate group of the Azene terminal. Chimera 8, which is an embodiment of the present invention, is a conjugate of Azene and Amonafide that exhibit an amine linking moiety that is more biostable than the carbamate linking moiety of Amonafidazene. A more biostable linking moiety secures, upon hydrolysis of the Azene terminal carbamate and subsequent self-immolative degradation of the p-aminobenzyl alcohol Azene moiety, the methylation in the proximity of the DNA intercalation site, suppressing the DNA repair processes. In conjugates such as Amonafidazene, the Azene moiety, which is linked to the drug moiety by a more biodegradable carbamate linking moiety, the DNA methylation part of the conjugate may be cleaved and detached from Amonafide moiety non-specifically, away from the DNA intercalation site, reducing the effectiveness of DNA methylation.

Hence, according to some embodiments of the present invention, the linking moiety is more biostable than a carbamate linking moiety. According to some embodiments, the linking moiety is more biostable than the terminal carbamate group of the Azene moiety. The following linking moieties are less labile under physiological (in vivo) conditions than carbamate: acetal, aldimine, amide, aminal, amine, aminoacetal, boroalkyl, boronate, carbonate, carboxylate, cycloalkene, cyclohexene, disulfide, ester, ether, heteroalicyclic, heteroaryl, hydrazone, imide, imine, ketal, ketimine, lactam, lactone, oxime, phosphate ester, phosphine, phosphite, phosphonate, semicarbazone, sulphate, sulphone, thioacetal, thioether, thioketal, triazine, and triazole.

According to embodiments of the present invention, the linking moiety is not carbamate, for reasons presented hereinabove.

According to some embodiments of the present invention, the linking moiety is an amino group, as defined hereinbelow.

The positions at which the bioactive agent is linked to the Azene moiety are generally selected such that once cleaved, any remaining moiety stemming from the linking moiety on the bioactive agent, if at all, does not substantially preclude its biological activity (mechanism of biological activity). Suitable positions depend on the type of bioactive agent. According to some embodiments of the present invention, the linking moieties are form such that the biological activity of the bioactive agent, once released from the Azene moiety, is not abolished and remains substantially the same as the biological activity of a similar pristine bioactive agent.

The phrase “reactive group”, as used herein, refers to a chemical group that is capable of undergoing a chemical reaction that typically leads to the formation a covalent bond. Chemical reactions that lead to a bond formation include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a reactive group.

Representative examples of reactive groups include, without limitation, acyl halide, aldehyde, alkoxy, alkyne, amide, amine, aryloxy, azide, aziridine, azo, carbonyl, carboxyl, carboxylate, cyano, diene, dienophile, epoxy, guanidine, guanyl, halide, hydrazide, hydrazine, hydroxy, hydroxylamine, imino, isocyanate, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

According some embodiments of the present invention, various elements of the Azene moiety presented herein are attached to one or more linking moieties via spacer moieties. As used herein, the phrase “spacer moiety” describes a chemical moiety that typically extends between two chemical moieties and is attached to each of the chemical moieties via covalent bonds. The spacer moiety may be linear or cyclic, be branched or unbranched, rigid or flexible.

The nature of the spacer moieties can be regarded as having an effect on two aspects, the synthetic aspect, namely the influence of the spacer moieties on the process of preparing the Azene moiety, and the influence of the spacer moieties on the biology activity of the Azene moiety in terms of biological activity (i.e., methylation), bioavailability and other ADME-Tox considerations.

According to some embodiments of the present invention, the spacer moieties are selected such that they allow and/or promote the conjugation reaction between the Azene moiety and the bioactive agent, and reduce the probability for the formation of side-products due to undesired reactions. Such traits can be selected for in terms of spacer's length, flexibility, structure and specific chemical reactivity or lack thereof. Spacer moieties with fewer reactive groups will present a simpler synthetic challenge, requiring less protection/deprotection steps and affording higher chemical yields. For example, saturated and linear alkyls of 1-10, or 1-5 carbon atoms, having one reactive group at the end atom for conjugation with a corresponding reactive group, would afford substantially higher yield and fewer side products. Similarly, a spacer moiety based on one or two chained benzyl rings would also lead to an efficient conjugation reaction.

According to some embodiments of the present invention, the spacer moieties are selected such that they provide favorable cleavage conditions, as these are discussed hereinbelow. For example, a spacer may alter the accessibility of an enzyme to the linking moiety, thereby allowing the enzyme to cleave the linkage between the bioactive agent and the Azene moiety.

According to some embodiments of the present invention, the spacer moieties include, without limitation, —CH₂—, —CH₂—O—, —(CH₂)₂—, —(CH₂)₂—O—, —(CH₂)₃—, —(CH₂)₃—O—, —(CH₂)₄—, —(CH₂)₅—, —(CH₂)₆—, —(CH(CH₃))—CH₂—, —CH═CH—CH═CH—, —C≡C—C≡C—, —CH₂CH(OH)CH₂—, —CH₂—O—CH₂—, —CH₂—O—CH₂—O—, —(CH₂)₂—O—(CH₂)₂—, —(CH₂)₂—O—(CH₂)₂—O—, —CH₂-mC₆H₄—CH₂—, —CH₂-mC₆H₄—CH₂—O—, —CH₂-pC₆H₄—CH₂—, —CH₂-pC₆H₄—CH₂—O—, —CH₂—NHCO—, —C₆H₄—NHCO—, —CH₂—O—CH₂— and —CH═CH—CH₂—NH—(CH₂)₂—.

In some embodiments, a spacer moiety can be regarded as forming a part of a linking moiety.

Examples of linking moieties, according to some embodiments of the present invention, include without limitation, amine (N; secondary and tertiary), ether (O), thioether (S), amide, carbonate, lactone, lactam, carboxylate, ester, boroalkyl, boronate, sulphone, sulphate, phosphonate, phosphine, phosphite, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein.

According to some embodiments, the linking moiety is selected from the group consisting of:

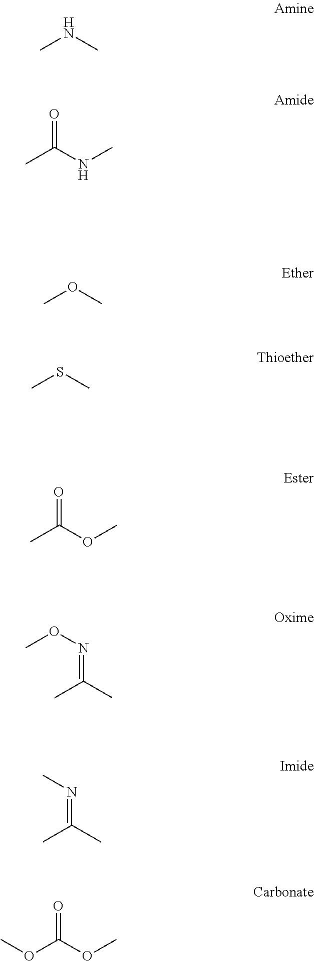

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

### Exemplary Conjugates:

Table 1 below presents non-limiting examples of conjugates, according to some embodiments of the present invention, some of which have been prepared and tested for anticancer activity as a proof of the general concept of the present invention. Each of the compounds presented herein is an independent embodiment of the present invention.

TABLE 1

| Conjugate | Structure | Conjugated drug |
|---|---|---|
| Chimera 1 | 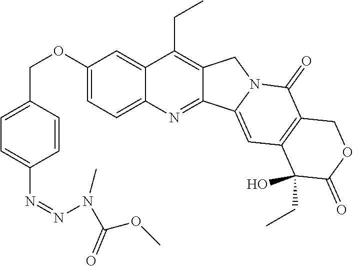 [SMILES: CCc1c2c(nc3ccc(OCc4ccc(/N=N\N(C)C(=O)OC)cc4)cc13)-c1cc3c(c(=O)n1C2)COC(=O)[C@]3(O)CC] | SN-38 |
| Chimera 2 | 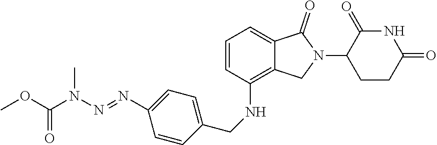 [SMILES: COC(=O)N(C)/N=N/c1ccc(CNc2cccc3c2CN(C2CCC(=O)NC2=O)C3=O)cc1] | Lenalidomide |
| Chimera 3 | 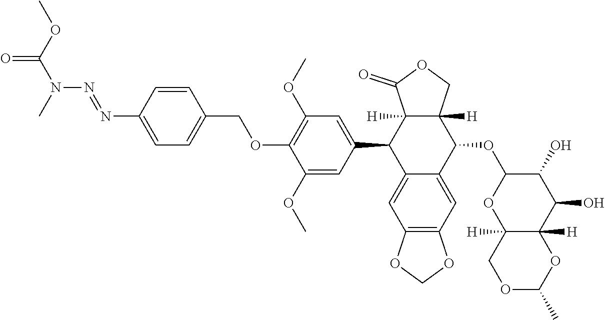 [SMILES: COC(=O)N(C)/N=N/c1ccc(COc2c(OC)cc([C@@H]3c4cc5c(cc4[C@@H](OC4O[C@@H]6CO[C@@H](C)O[C@H]6[C@H](O)[C@H]4O)[C@H]4COC(=O)[C@H]34)OCO5)cc2OC)cc1] | Etoposide |
| Chimera 4 | 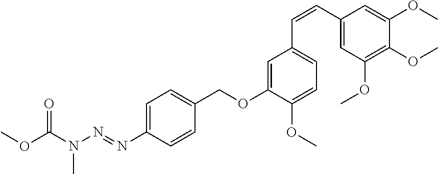 | Combretastatin |
| Chimera 5 | 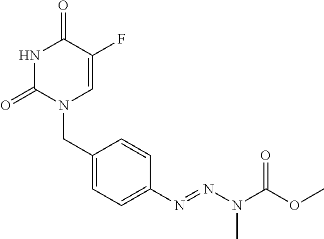 [SMILES: COC(=O)N(C)/N=N/c1ccc(Cn2cc(F)c(=O)[nH]c2=O)cc1] | 5-Fluorouracil |
| Chimera 6 | 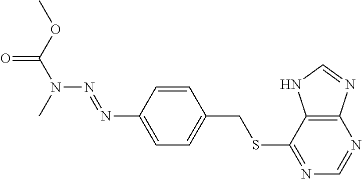 [SMILES: COC(=O)N(C)/N=N/c1ccc(CSc2ncnc3nc[nH]c23)cc1] | 6-Mercaptopurine |
| Chimera 7 | 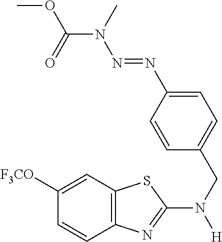 [SMILES: COC(=O)N(C)/N=N/c1ccc(CNc2nc3ccc(OC(F)(F)F)cc3s2)cc1] | Riluzole |
| Chimera 8 | 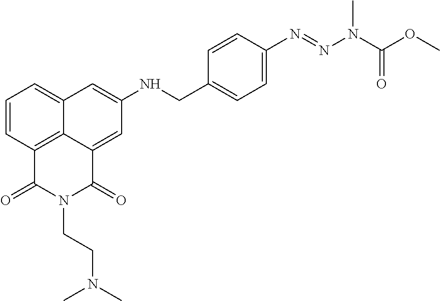 [SMILES: COC(=O)N(C)/N=N/c1ccc(CNc2cc3c4c(cccc4c2)C(=O)N(CCN(C)C)C3=O)cc1] | Amonafide (DW-397) |
| Chimera 9 | 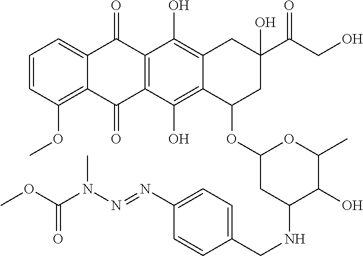 | Doxorubicin |
| Chimera 10 | 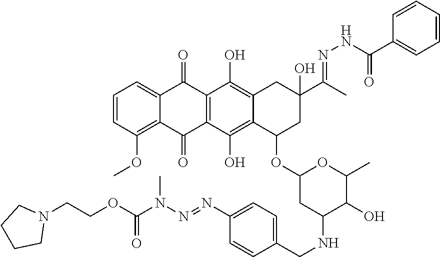 [SMILES: COc1cccc2c1C(=O)c1c(O)c3c(c(O)c1C2=O)CC(O)(/C(C)=N/NC(=O)c1ccccc1)CC3OC1CC(NCc2ccc(/N=N/N(C)C(=O)OCCN3CCCC3)cc2)C(O)C(C)O1] | Zorubicin |
| Chimera 11 | 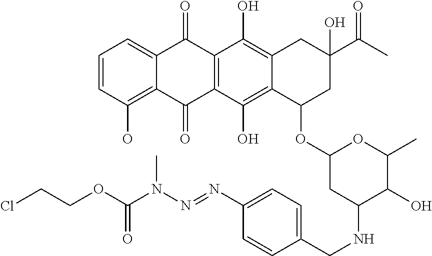 [SMILES: CC(=O)C1(O)Cc2c(O)c3c(c(O)c2C(OC2CC(NCc4ccc(/N=N/N(C)C(=O)OCCCl)cc4)C(O)C(C)O2)C1)C(=O)c1c(O)cccc1C3=O] | Carubicin |
| Chimera 12 | 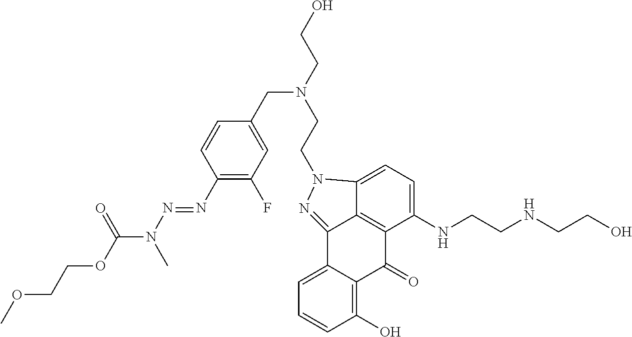 [SMILES: COCCOC(=O)N(C)/N=N/c1ccc(CN(CCO)CCn2nc3c4c(c(NCCNCCO)ccc42)C(=O)c2c(O)cccc2-3)cc1F] | Losoxantrone |
| Chimera 13 | 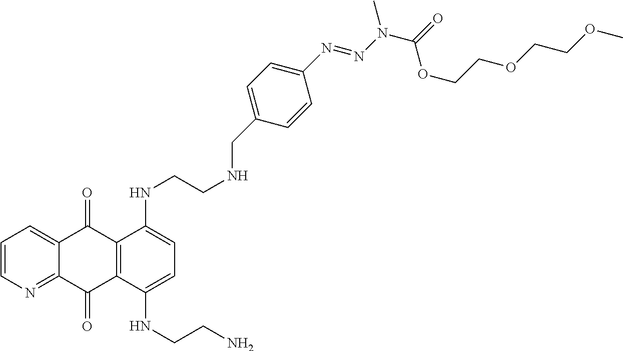 [SMILES: COCCOCCOC(=O)N(C)/N=N/c1ccc(CNCCNc2ccc(NCCN)c3c2C(=O)c2cccnc2C3=O)cc1] | Pixantrone |
| Chimera 14 | 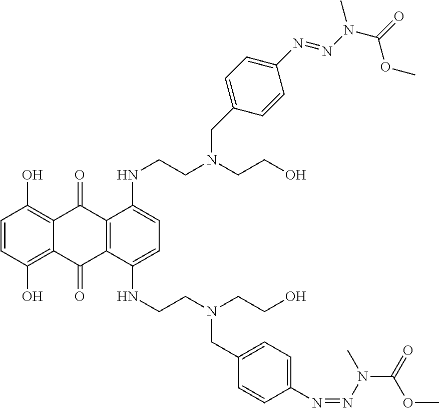 [SMILES: COC(=O)N(C)/N=N\c1ccc(CN(CCO)CCNc2ccc(NCCN(CCO)Cc3ccc(/N=N/N(C)C(=O)OC)cc3)c3c2C(=O)c2c(O)ccc(O)c2C3=O)cc1] | Mitoxantrone |
| Chimera 15 | 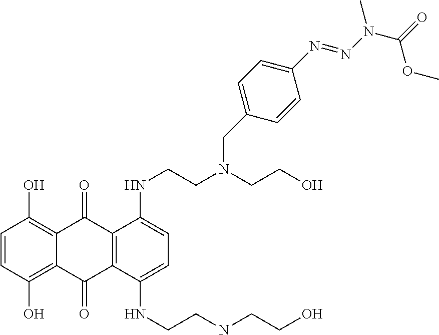 [SMILES: COC(=O)N(C)/N=N/c1ccc(CN(CCO)CCNc2ccc(NCCNCCO)c3c2C(=O)c2c(O)ccc(O)c2C3=O)cc1] | Mitoxantrone |
| Chimera 16 | 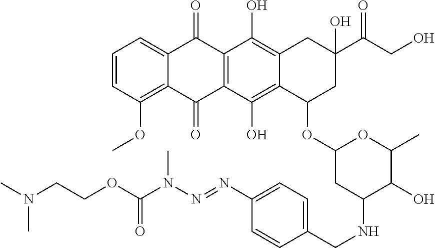 [SMILES: COc1cccc2c1C(=O)c1c(O)c3c(c(O)c1C2=O)CC(O)(C(=O)CO)CC3OC1CC(NCc2ccc(/N=N/N(C)C(=O)OCCN(C)C)cc2)C(O)C(C)O1] | Doxorubicin |
| Chimera 17 | 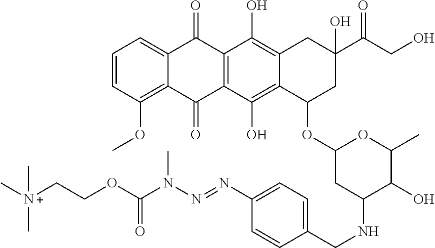 [SMILES: COc1cccc2c1C(=O)c1c(O)c3c(c(O)c1C2=O)CC(O)(C(=O)CO)CC3OC1CC(NCc2ccc(/N=N/N(C)C(=O)OCC[N+](C)(C)C)cc2)C(O)C(C)O1] | Doxorubicin |
| Chimera 18 | 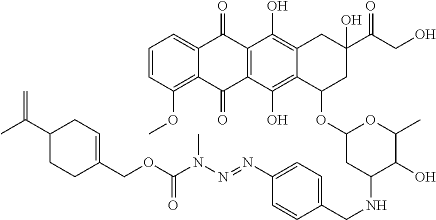 [SMILES: C=C(C)C1CC=C(COC(=O)N(C)/N=N/c2ccc(CNC3CC(OC4CC(O)(C(=O)CO)Cc5c(O)c6c(c(O)c54)C(=O)c4c(OC)cccc4C6=O)OC(C)C3O)cc2)CC1] | Doxorubicin |
| Chimera 19 | 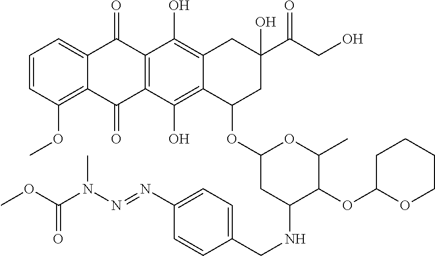 [SMILES: COC(=O)N(C)/N=N/c1ccc(CNC2CC(OC3CC(O)(C(=O)CO)Cc4c(O)c5c(c(O)c43)C(=O)c3c(OC)cccc3C5=O)OC(C)C2OC2CCCCO2)cc1] | Pirarubicin |
| Chimera 20 | 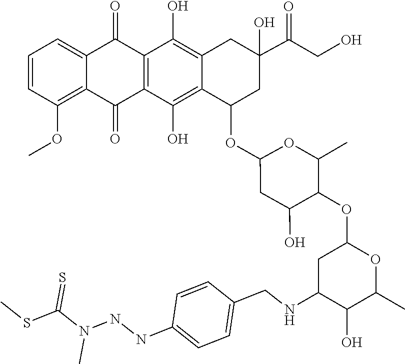 | Sabarubicin |
| Chimera 21 | 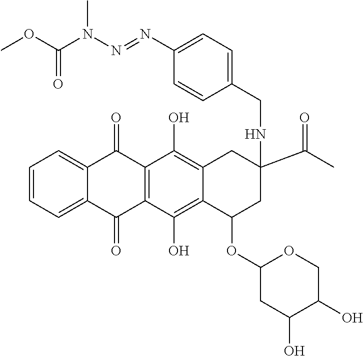 [SMILES: COC(=O)N(C)/N=N/c1ccc(CNC2(C(C)=O)Cc3c(O)c4c(c(O)c3C(OC3CC(O)C(O)CO3)C2)C(=O)c2ccccc2C4=O)cc1] | Amrubicin |
| Chimera 22 | 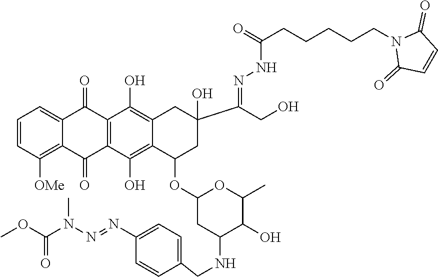 [SMILES: COC(=O)N(C)/N=N/c1ccc(CNC2CC(OC3CC(O)(/C(CO)=N/NC(=O)CCCCCN4C(=O)C=CC4=O)Cc4c(O)c5c(c(O)c43)C(=O)c3c(OC)cccc3C5=O)OC(C)C2O)cc1] | Aldoxorubicin |
| Chimera 23 | 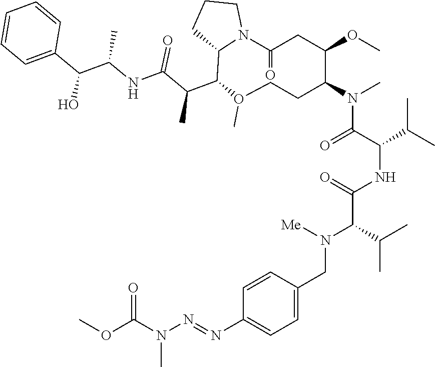 | Monomethyl auristatin E (MMAE) |

### Anthracycline Conjugates:

Anthracyclines are a class of chemotherapy drugs that are used to treat a wide variety of cancers, including breast cancer, leukemia, lymphoma, and sarcoma. They are among the most effective anticancer drugs ever developed, but they can also have serious side effects, including heart damage. Anthracyclines are derived from bacteria and have a unique chemical structure that allows them to interact with DNA and RNA. This interaction prevents cancer cells from dividing and growing. Anthracyclines are typically given by injection into a vein, but they can also be given directly into the bladder or into a tumor. They are often used in combination with other chemotherapy drugs.

Non-limiting examples of anthracyclines include Doxorubicin (Adriamycin), Daunorubicin (Daunomycin), Epirubicin (Ellence), Idarubicin (Idamycin), Annamycin, Camsirubicin, Berubicin, Daunorubicin (Daunomycin) and Daunomustine, the structures of which are presented below:

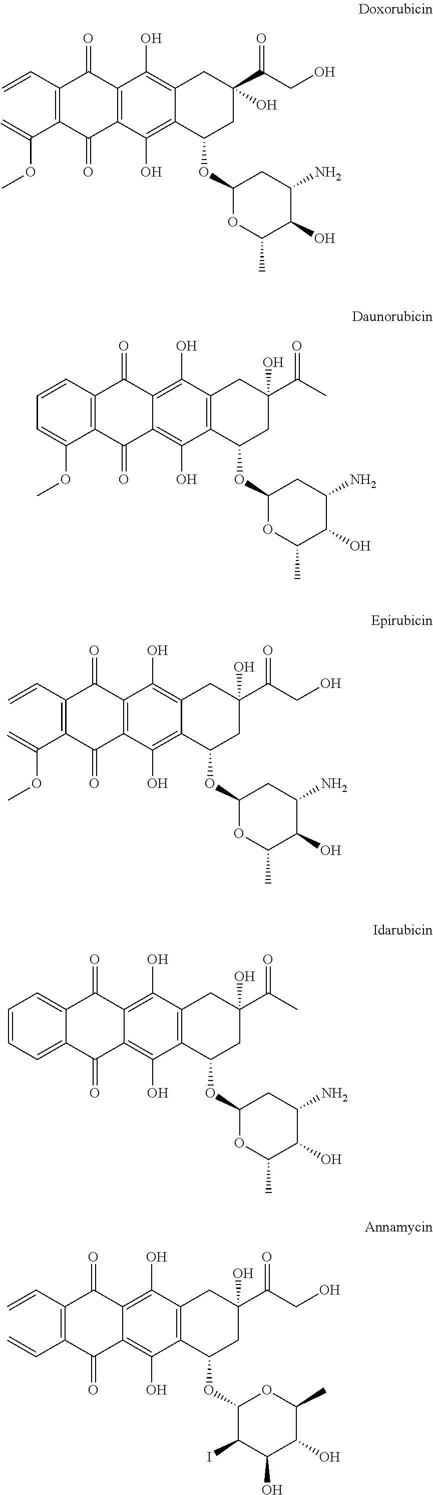

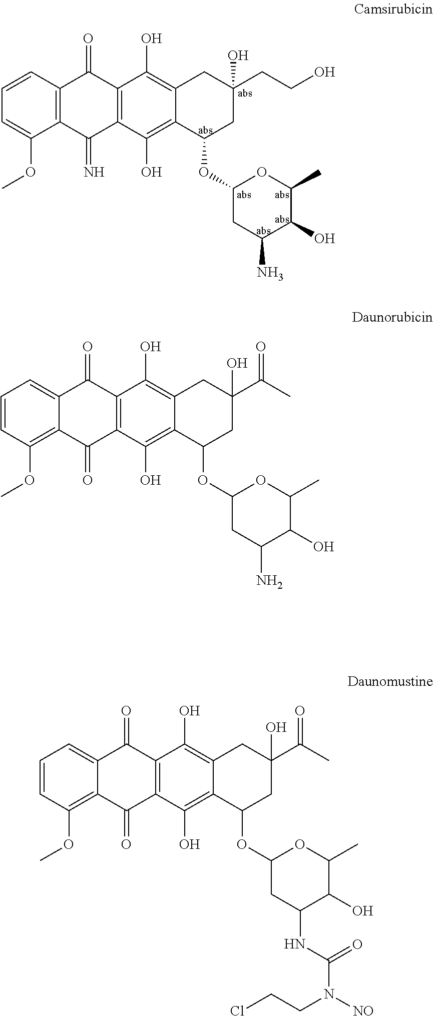

According to some embodiment of the present invention, the anticancer drug is an anthracycline. In some respective embodiments, the linking moiety (L in Formula I) is amine.

Thus, according to some embodiments of the present invention, there is provided a compound, characterized by general Formula II:

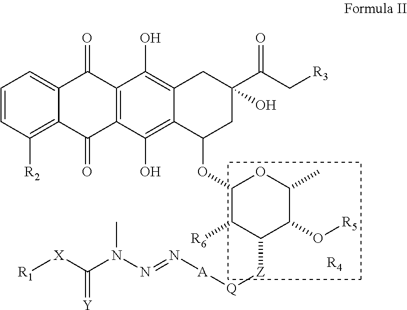

wherein:
R₁ is selected from the group consisting of hydrogen, alkyl, alkoxyalkyl, alkoxy, aryl, heteroaryl, alkylaryl, o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, a tertiary alkyl amine, a quaternary ammonium choline, L-valine-L-citrulline-p-aminobenzyl alcohol (Val-Cit-PABA), L-valine-L-alanine-p-aminobenzyl alcohol (Val-Ala-PABA), or a protecting group;
each of R₂ and R₃ is independently selected from the group consisting of hydrogen, hydroxy, alkyl (methyl, ethyl, propyl, etc.), alkoxyalkyl (-alkyl-O-alkyl, methoyalkyl, methoxyethyl, etc.), alkoxy (—O-alkyl, methoxy, ethoxy, etc.), aryl/heteroaryl (phenyl, pyridine, etc.), alkylaryl (benzyl), o-nitrobenzyl, benzhydryl, trityl, alkylsilyl, and the likes or a protecting group;
R₄ is selected from the group consisting of the (2R,4S,5S,6S), (2R,4S,5R,6S), (2R,4S,5S,6S) and (2R,4S,5S,6S) stereoisomer of 4-(λ¹-azaneyl)-2-methyl-6-(λ²-oxidaneyl)tetrahydro-2H-pyran-3-ol;
R₅ is selected from the group consisting of hydrogen, alkyl, aryl, heteroaryl and benzyl;
R₆ is selected from the group consisting of hydrogen, F, Cl, Br and I;
Z is selected from the group consisting of O, S, NH and N-alkyl;
Q is selected from the group consisting of (CH₂)₁₋₁₀, C═O, C═NH, C═N-alkyl, C═N-aryl and C═S;
A is an aromatic or a heteroaromatic ring; and
each of X and Y is independently O, S, N, NH, alkylene (C₁₋₅), sulfone, and phosphonate or forming a triazene protecting group.

In some embodiments, the anthracycline conjugates are represented by the general formula II(1):

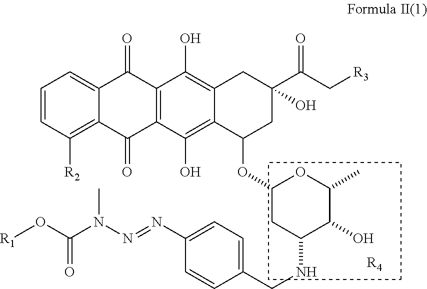

wherein:
each of R₁, R₂ and R₃ is independently selected from the group consisting of hydrogen, hydroxy, alkyl (methyl, ethyl, propyl, etc.), alkoxyalkyl (-alkyl-O-alkyl, methoyalkyl, methoxyethyl, etc.), alkoxy (—O-alkyl, methoxy, ethoxy, etc.), aryl/heteroaryl (phenyl, pyridine, etc.), alkylaryl (benzyl), ortho nitrobenzyl, benzhydryl, trityl, alkylsilyl, and the likes or a protecting group; and
R₄ is selected from the group consisting of the (2R,4S,5S,6S), (2R,4S,5R,6S), (2R,4S,5S,6S) and (2R,4S,5S,6S) stereoisomer of 4-(λ¹-azaneyl)-2-methyl-6-(λ²-oxidaneyl)tetrahydro-2H-pyran-3-ol.

Table 2 below presents some exemplary conjugates, according to some embodiments of the present invention, that were prepared and tested for anticancer activity.

TABLE 2

| Conjugated drug | Structure | IUPAC |
|---|---|---|
| Chimera 9 (Doxorubizen) | 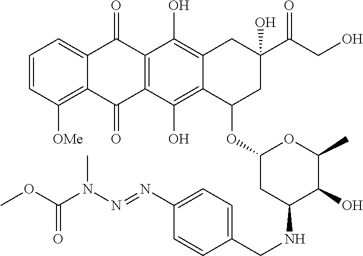 [SMILES: COC(=O)N(C)/N=N/c1ccc(CN[C@H]2C[C@H](OC3C[C@](O)(C(=O)CO)Cc4c(O)c5c(c(O)c43)C(=O)c3c(OC)cccc3C5=O)O[C@@H](C)[C@H]2O)cc1] | methyl (E)-3-(4-((((2S,3S,4S,6R)- 3-hydroxy-2-methyl-6-(((3S)- 3,5,12-trihydroxy-3-(2- hydroxyacetyl)-10-methoxy-6,11- dioxo-1,2,3,4,6,11- hexahydrotetracen-1- yl)oxy)tetrahydro-2H-pyran-4- yl)amino)methyl)phenyl)-1- methyltriaz-2-ene-1-carboxylate |
| Chimera 24 (Epirubizen) | 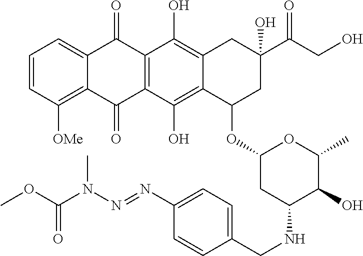 [SMILES: COC(=O)N(C)/N=N/c1ccc(CN[C@@H]2C[C@H](OC3C[C@](O)(C(=O)CO)Cc4c(O)c5c(c(O)c43)C(=O)c3c(OC)cccc3C5=O)O[C@H](C)[C@H]2O)cc1] | methyl (E)-3-(4-((((2R,3S,4R,6R)- 3-hydroxy-2-methyl-6-(((3S)- 3,5,12-trihydroxy-3-(2- hydroxyacetyl)-10-methoxy-6,11- dioxo-1,2,3,4,6,11- hexahydrotetracen-1- yl)oxy)tetrahydro-2H-pyran-4- yl)amino)methyl)phenyl)-1- methyltriaz-2-ene-1-carboxylate |
| Chimera 25 (Daunorubizen) | 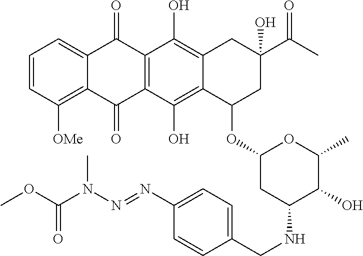 [SMILES: COC(=O)N(C)/N=N/c1ccc(CN[C@@H]2C[C@H](OC3C[C@](O)(C(C)=O)Cc4c(O)c5c(c(O)c43)C(=O)c3c(OC)cccc3C5=O)O[C@H](C)[C@@H]2O)cc1] | methyl (E)-3-(4-((((2R,3R,4R,6R)- 6-(((3S)-3-acetyl-3,5,12- trihydroxy-10-methoxy-6,11-dioxo- 1,2,3,4,6,11-hexahydrotetracen-1- yl)oxy)-3-hydroxy-2- methyltetrahydro-2H-pyran-4- yl)amino)methyl)phenyl)-1- methyltriaz-2-ene-1-carboxylate |
| Chimera 26 (Idarubizen) | 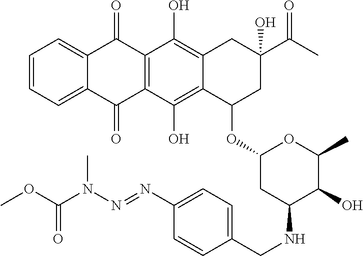 [SMILES: COC(=O)N(C)/N=N/c1ccc(CN[C@H]2C[C@H](OC3C[C@](O)(C(C)=O)Cc4c(O)c5c(c(O)c43)C(=O)c3ccccc3C5=O)O[C@@H](C)[C@H]2O)cc1] | methyl (E)-3-(4-((((2S,3S,4S,6R)- 6-(((3S)-3-acetyl-3,5,12- trihydroxy-6,11-dioxo-1,2,3,4,6,11- hexahydrotetracen-1-yl)oxy)-3- hydroxy-2-methyltetrahydro-2H- pyran-4-yl)amino)methyl)phenyl)- 1-methyltriaz-2-ene-1-carboxylate |
| Chimera 27 (Doxorubicin + Azene) | 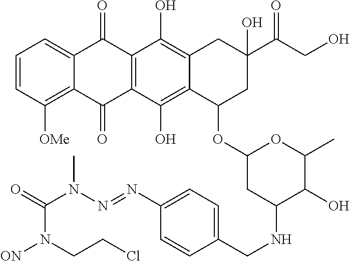 | (E)-N-(2-chloroethyl)-3-(4-(((3- hydroxy-2-methyl-6-((3,5,12- trihydroxy-3-(2-hydroxyacetyl)-10- methoxy-6,11-dioxo-1,2,3,4,6,11- hexahydrotetracen-1- yl)oxy)tetrahydro-2H-pyran-4- yl)amino)methyl)phenyl)-1-methyl- N-nitrosotriaz-2-ene-1- carboxamide |
| Chimera 28 (Doxorubicin + Azene) | 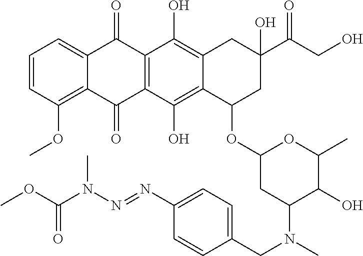 | methyl (E)-3-(4-(((3-hydroxy-2- methyl-6-((3,5,12-trihydroxy-3-(2- hydroxyacetyl)-10-methoxy-6,11- dioxo-1,2,3,4,6,11- hexahydrotetracen-1- yl)oxy)tetrahydro-2H-pyran-4- yl)(methyl)amino)methyl)phenyl)- 1-methyltriaz-2-ene-1-carboxylate |
| Chimera 29 (Doxorubicin + Azene) | 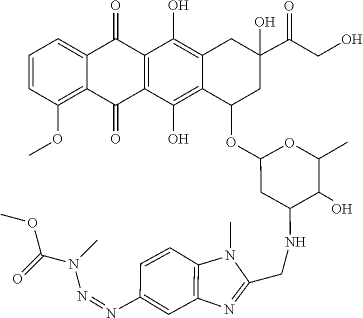 [SMILES: COC(=O)N(C)/N=N\c1ccc2c(c1)nc(CNC1CC(OC3CC(O)(C(=O)CO)Cc4c(O)c5c(c(O)c43)C(=O)c3c(OC)cccc3C5=O)OC(C)C1O)n2C] | methyl (Z)-3-(2-(((3-hydroxy-2- methyl-6-((3,5,12-trihydroxy-3-(2- hydroxyacetyl)-10-methoxy-6,11- dioxo-1,2,3,4,6,11- hexahydrotetracen-1- yl)oxy)tetrahydro-2H-pyran-4- yl)amino)methyl)-1-methyl-1H- benzo[ d] imidazol-5-y1)-1- methyltriaz-2-ene-1-carboxylate |
| Chimera 30 (Doxorubicin + Azene) | 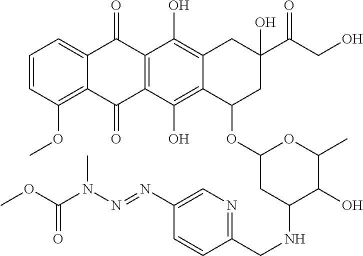 | methyl (E)-3-(6-(((3-hydroxy-2- methyl-6-((3,5,12-trihydroxy-3-(2- hydroxyacetyl)-10-methoxy-6,11- dioxo-1,2,3,4,6,11- hexahydrotetracen-1- yl)oxy)tetrahydro-2H-pyran-4- yl)amino)methyl)pyridin-3-yl)-1- methyltriaz-2-ene-1-carboxylate |

### Anticancer Drugs

Anticancer drugs, which are contemplated as bioactive agent in the context of the present invention as conjugates drugs, include, without limitation, Acivicin, Aclarubicin, Acodazole Hydrochloride, Acronine, Adozelesin, Adriamycin, Aldesleukin, Aldoxorubicin, Altretamine, Ambomycin, Ametantrone Acetate, Aminoglutethimide, Amonafide, Amrubicin, Amsacrine, Anastrozole, Anthramycin, Asparaginase, Asperlin, Azacitidine, Azetepa, Azotomycin, Batimastat, Benzodepa, Bicalutamide, Bisantrene, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin, Carzelesin, Cedefingol, Chlorambucil, Cirolemycin, Cisplatin, Cladribine, Combretastatin, Crisnatol Mesylate, Cyclophosphamide, Cytarabine, Dacarbazine, Dactinomycin, Daunorubicin, Decitabine, Dexormaplatin, Dezaguanine, Dezaguanine Mesylate, Diaziquone, Docetaxel, Doxorubicin, Droloxifene, Droloxifene Citrate, Dromostanolone Propionate, Duazomycin, Edatrexate, Eflornithine, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Etoposide, Etoposide Phosphate, Etoprine, Fadrozole, Fazarabine, Fenretinide, Floxuridine, Fludarabine Phosphate, 5-Fluorouracil, Flurocitabine, Fosquidone, Fostriecin Sodium, Gemcitabine, Hydroxyurea, Idarubicin, Ifosfamide, Ilmofosine, Interferon Alfa-2a, Interferon Alfa-2b, Interferon Alfa-n1, Interferon Alfa-n3, Interferon Beta-Ia, Interferon Gamma-I b, Iproplatin, Irinotecan Hydrochloride, Lanreotide Acetate, Lenalidomide, Letrozole, Leuprolide Acetate, Liarozole Hydrochloride, Lometrexol Sodium, Lomustine, Losoxantrone, Losoxantrone Hydrochloride, Masoprocol, Maytansine, Mechlorethamine Hydrochloride, Megestrol Acetate, Melengestrol Acetate, Melphalan, Menogaril, 6-Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone, Mitoxantrone, Monomethyl auristatin E (MMAE), Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Pirarubicin, Piroxantrone Hydrochloride, Pixantrone, Plicamycin, Plomestane, Porfimer Sodium, Porfiromycin, Prednimustine, Procarbazine Hydrochloride, Puromycin, Puromycin Hydrochloride, Pyrazofurin, Riboprine, Riluzole, Rogletimide, Sabarubicin, Safingol, Safingol Hydrochloride, Semustine, Simtrazene, Sparfosate Sodium, Sparsomycin, Spirogermanium, Spiromustine, Spiroplatin, Streptonigrin, Streptozocin, Sulofenur, Talisomycin, Taxol, Tecogalan Sodium, Tegafur, Teloxantrone, Temoporfin, Teniposide, Teroxirone, Testolactone, Thiamiprine, Thioguanine, Thiotepa, Tiazofuirin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, and Zorubicin.

Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Approved chemotherapy agents, which are contemplated as bioactive agent in the context of the present invention as conjugates drugs, include, without limitation, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid.

### Pharmaceutical Composition:

According to some embodiments of the present invention, the compounds provided herein are contemplated in a form of a prodrug, an ester, a solvate, a hydrate and/or pharmaceutically acceptable salt thereof.

The phrase “pharmaceutically acceptable salt” refers to a charged species of the parent conjugate and its counter ion(s), which is typically used to modify the solubility characteristics of the parent conjugate and/or to reduce any significant irritation to an organism by the parent conjugate, while not abrogating the biological activity and properties of the administered conjugate.

Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional nontoxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stochiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, PA, 19143, p. 1418).

Representative examples of pharmaceutically acceptable salts that can be efficiently used in the context of the present invention include, without limitation, conjugate hydrochloride and conjugate mesylate.

The term “prodrug” refers to an agent, which is converted into a bioactive agent (the active parent drug) in vivo. In essence, the conjugates presented herein constitute a form of a prodrug, as drug moieties, which are designed for release as bioactive agents in a controllable manner, are linked thereto. Prodrugs are typically useful for facilitating and/or targeting the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of a bioactive agent in vivo. An example, without limitation, of a prodrug would be a bioactive agent, according to some embodiments of the present invention, having one or more carboxylic acid moieties, which is administered as an ester (the “prodrug”). Such a prodrug is hydrolyzed in vivo, to thereby provide the free bioactive agent (the parent drug). The selected ester may affect both the solubility characteristics and the hydrolysis rate of the prodrug. A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)acyl (e.g., acetyl) group to form an ester group, and/or any one or more of the carboxylic acid groups of the compound is modified by an alkoxy or aryloxy group, optionally (C₁₋₄)alkoxy (e.g., methyl, ethyl) group to form an ester group.

According to some embodiments of the present invention, the compounds provided herein are in a form of a salt, preferably a pharmaceutically acceptable salt, or in the form of an ester (i.e., a prodrug), preferably a methyl ester. Further preferably the compounds provided herein are in a form of a salt or an ester of a carboxy group functionality.

The term “solvate” refers to a complex of variable stoichiometry (e.g., di-, tri-, tetra-, penta-, hexa-, and so on), which is formed by a solute (the compound as described herein) and a solvent, whereby the solvent does not interfere with the biological activity of the solute. Suitable solvents include, for example, ethanol, acetic acid and the like. The term “hydrate” refers to a solvate, as defined hereinabove, where the solvent is water.

Since the conjugates presented herein carry, deliver and controllably release a wide variety of drugs, the conjugates can be used to treat various medical conditions, including cancer. The conjugates presented herein can therefore be used as an active ingredient in a variety of pharmaceutical compositions, and in the preparation of a variety of medicaments.

Accordingly, there is provided a pharmaceutical composition that includes, as an active ingredient, the conjugate, according to embodiments of the present invention, and a pharmaceutically acceptable carrier. Similarly, there is provided a use of the conjugate, according to embodiments of the present invention, in the preparation of a medicament.

According to some embodiments of the present invention, the pharmaceutical composition or medicament, are used to treat a medical condition.

In any of the embodiments described herein relating to in vivo use, the compound, according to any of the respective embodiments described herein, may optionally be administered to an organism per se, or in a form of a pharmaceutical composition which may optionally further comprise suitable carriers or excipients.

As used herein a “pharmaceutical composition” refers to a preparation of one or more of the active ingredients described herein (including diagnostic and/or imaging agents) with other chemical components such as physiologically suitable carriers and excipients. The purpose of a pharmaceutical composition is to facilitate administration of a compound to an organism (e.g., for a therapeutic and/or diagnostic application).

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of the compound, according to any of the aspects of embodiments of the invention described herein, may be found in “Remington's Pharmaceutical Sciences,” Mack Publishing Co., Easton, PA, latest edition, which is incorporated herein by reference.

Suitable routes of administration may, for example, local or systemic routes, and include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Local administration may optionally be effected, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethylcellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

In some of any of the embodiments described herein, the carrier is or comprises a cyclodextrin.

Pharmaceutical compositions suitable for use in context of some embodiments of the invention include compositions wherein the active ingredients are contained in an amount effective to achieve the intended purpose (e.g., a therapeutic and/or diagnostic purpose). More specifically, an effective amount means an amount of a complex described herein sufficient to effect a method and/or imaging technique described herein, or (e.g., in radiation therapy) to prevent, alleviate or ameliorate symptoms of a treated disorder (e.g., a benign or malignant tumor) or prolong the survival of the subject being treated.

Determination of an effective amount is well within the capability of those skilled in the art (e.g., based on the expected background signal of a given imaging technique, which will be known to one skilled in the art pertaining to the imaging technique), especially in light of the detailed disclosure provided herein.

For any preparation used in the methods of the invention, the effective amount or dose can be estimated initially from in vitro and cell culture assays. For example, a dose can be formulated in animal models to achieve a desired concentration or titer. Such information can be used to more accurately determine useful doses in humans.

Toxicity and effective amounts of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide cells (e.g., tumor cells) levels of the active ingredient that are sufficient (e.g., in radiation therapy) to induce or suppress the biological effect (minimal effective concentration, MEC), or to distinguish the cells (e.g., in hypoxic tissue) from surrounding cells. The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated (according to any of the respective embodiments described herein), dosing can be of a single or a plurality of administrations, with course of treatment lasting from several days to several weeks or until cure is effected or diminution of the disease state is achieved.

The amount of a composition to be administered will, of course, be dependent on the subject being treated or imaged, the type of imaging technique, the severity of the affliction, the manner of administration, the judgment of the prescribing physician, etc.

Compositions of some embodiments of the invention (according to any of the aspects described herein) may, if desired, be presented in a pack or dispenser device, such as an FDA approved kit, which may contain one or more unit dosage forms containing complex described herein. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceutical and diagnostic agents, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated use or method (according to any of the respective embodiments described herein).

According to some embodiments, the composition is packaged in a packaging material and identified in print, in or on the packaging material, for use in the treatment of a medical condition, e.g., cancer, treatable by at least one of the drugs linked and controllably releasable from the conjugate.

### Methods and Uses:

Also provided herein is a method of treating a medical condition in a subject in need thereof, which includes administering to the subject a therapeutically effective amount of the conjugate or a composition comprising the same, according to embodiments of the present invention.

As used herein, the phrase “therapeutically effective amount” describes an amount of an active agent or a conjugate being administered, which will relieve to some extent one or more of the symptoms of the medical condition being treated. In the context of the present embodiments, the phrase “therapeutically effective amount” describes an amount of a conjugate being administered and/or re-administered, which will relieve to some extent one or more of the symptoms of the condition being treated by being at a level that is harmful to the target cell(s) or microorganism(s), and cause a disruption to the life-cycle of the target cell(s) or microorganism(s).

In the context of embodiments of the present invention, the therapeutically effective amount may refer to the conjugate as a whole or to the amount of one or more bioactive agent releasably attached thereto. The efficacy of any bioactive agent, including the conjugates presented herein, can be determined by several methodologies known in the art.

According to another aspect of embodiments of the present invention, any one of the conjugates described herein is identified for use in treating a subject diagnosed with a medical condition treatable by at least one of the drugs linked and controllably releasable from the conjugate.

According to another aspect of embodiments of the present invention, there is provided a use of any of the conjugates described herein as a medicament. In some embodiments, the medicament is for treating a subject diagnosed with a medical condition treatable by at least one of the drugs linked and controllably releasable from the conjugate.

In any of the methods and uses described herein, the conjugate can be administered as a part of a pharmaceutical composition, which further comprises a pharmaceutical acceptable carrier, as detailed herein. The carrier is selected suitable to the selected route of administration.

According to some embodiments of the present invention, the compositions, uses and method of treatment, according to some embodiment of the present invention, may include the co-administration of at least one additional therapeutically active agent, as this is defined and discussed herein.

### Cancer Treatment and Chemotherapy:

The conjugate presented herein can be used to treat any medical condition that is treatable by administration of a bioactive agent (drug). According to some embodiments of the present invention, it is advantageous to use the conjugate to treat medical conditions, which are treatable by administration of a combination of drugs.

In some embodiments of the present invention, the medical condition is associated with malignant cells and tumors, collectively referred to herein as cancer.

Cancer is a spontaneous, acquired or genetic disease in which mutations violate cell growth and survival pathways. Essentially abnormal tissue growth (neoplasm) develops through a process whereby cancer begins in a single cell and passes its malignant potential to subsequent generations of cells. A carcinogenic event is usually operated by some external disruptive factors, such as viruses, radiation (such as sunlight, x-rays and radioactive sources which emit energy and subatomic particles) and chemical carcinogens, mutagens or teratogens. Mammalian cells have multiple safeguards to protect them against the potentially lethal effects of cancer gene mutations, but when several genes are defective, an invasive cancer develops. Human cancers originate from mutations that usually occur in somatic tissues; however, hereditary forms of cancer exist in which individuals are heterozygous for a germline mutation.

The mutations target three types of genes (cancer genes), namely tumor suppressor genes, oncogenes, and stability genes. Loss-of-function mutations in tumor suppressors and gain-of-function mutations in oncogenes lead to cancer, while loss-of-function mutations in stability genes increase the rates of mutation of tumor suppressors and oncogenes. All cancer mutations operate similarly at the physiologic level: they drive the carcinogenic process by increasing tumor cell number through the stimulation of cell birth or the inhibition of cell-cycle arrest or cell death. The increase is usually caused by facilitating the provision of nutrients through enhanced angiogenesis, by activating genes that drive the cell cycle or by inhibiting normal apoptotic processes.

The most common types of cancer treatment are surgery, radiotherapy and chemotherapy. Radiotherapy is usually used alone or in combination with surgery and/or chemotherapy. Other types of treatments include hormone therapy that is used in combination with surgery and/or chemotherapy for treatment of, for example, androgen-dependent prostate cancer or estrogen-dependent breast cancer.

Cryosurgery uses cold liquid nitrogen or gas argon to destroy abnormal tissue. Relatively new additions to the family of cancer treatments include biological therapy and angiogenesis inhibitors. Biological therapy is based on the stimulation of the body's own immune system, either directly or indirectly, to fight off cancer or to diminish side effects caused by other treatments.

To date, chemotherapy remains the most common and most frequently used in cancer treatment, alone or in combination with other therapies. Currently available anticancer chemotherapies act by affecting specific molecular targets in proliferating cancer cells, leading to inhibition of essential intracellular processes such as DNA transcription, synthesis and replication.

Unfortunately, anticancerous drugs are highly toxic, as they are designed to kill mammalian cells, and are therefore harmful also to normal proliferating cells resulting in debilitating and even lethal side effects. Some of these adverse effects are gastrointestinal toxicity, nausea, vomiting, and diarrhea when the epithelial lining of the intestine is affected. Other side effects include alopecia, when the hair follicles are attacked, bone marrow suppression and neutropenia due to toxicity of hematopoietic precursors. Therefore, the effectiveness of currently used anticancerous drugs is dose-limited due to their toxicity to normal rapidly growing cells.

One of the contemporary approaches in the fight against cancer is engineering of molecular targeted drugs that permeate cancer cells and specifically modulate activity of molecules that belong to signal-transduction pathways. These targets include products of frequently mutated oncogenes, such as k-Ras and other proteins that belong to tyrosine kinase signal transduction pathways. For example, Imatinib (Gleevec®), is the first such drug, approved for treatment of chronic myelogenous leukemia (CML). Imatinib blocks the activity of non-receptor tyrosine kinase BCR-Able oncogene, present in 95% of patients with CML. Imatinib was found to be effective in the treatment of CML and certain tumors of the digestive tract. Nevertheless, as others, this new compound is not completely specific to its target; therefore, side effects emerge, including severe congestive cardiac failure, pulmonary tuberculosis, liver toxicity, sweet syndrome (acute febrile neutrophilic dermatosis), leukocytosis, dermal edemas, nausea, rash and musculoskeletal pain.

Angiogenesis inhibitors are currently investigated for their use in cancer treatment and to date, one anti-angiogenetic drug, Bevacizumab (Avastin®), was approved for the treatment of solid tumors in combination with standard chemotherapy. However, as in all chemotherapeutic drugs, Bevacizumab causes a number of adverse side effects such as hypertension, blood clots, neutropenia, neuropathy, proteinuria and bowel perforation.

In some embodiments, the targeting moiety of the conjugates presented herein, is responsible for the higher concentration of the conjugate at the targeted bodily site compared to non-targeted bodily sites, thereby reducing the adverse side effects associated with the toxicity of the anti-cancer drugs attached thereto. In addition, the linking moieties attached the anti-cancer drugs to the conjugate are selected such that they cleave in conditions that are present at the targeted site more so than in non-targeted sites, thereby releasing the payload of drugs at the targeted site at a higher rate compared to non-targeted sites.

Treatment of cancer is becoming even more complicated, since on top of the many factors that cause tumor formation and the multiple adverse side effects associated with currently available anticancerous agents, there are a myriad of mechanisms by which cells become resistant to unspecific drugs.

Mechanisms of drug resistance include prevention from entering the cells, pumping the drug out of the cells, enzymatic inactivation, prevention of drug activity by mutation or altered expression of the target, and inhibition of biochemical pathways by mutations in oncogenes, tumor-suppressor genes or stability genes.

Many of the most prevalent forms of human cancer resist effective chemotherapeutic intervention. Some tumor populations, especially adrenal, colon, jejunal, kidney and liver carcinomas, appear to have drug-resistant cells at the outset of treatment [Barrows, L. R., “Antineoplastic and Immunoactive Drugs”, Chapter 75, pp 1236-1262, in: Remington: The Science and Practice of Pharmacy, Mack Publishing Co. Easton, Pa., 1995]. In other cases, a resistance-conferring genetic change occurs during treatment; the resistant daughter cells then proliferate in the environment of the drug. Whatever the cause, resistance often terminates the usefulness of an anticancerous drug, and the emergence of multidrug resistance (MDR) sadly lead to therapeutic failure in many cancer patients [Liscovitch, M. and Lavie, Y., IDrugs, 2002, 5(4), 349-55].

Many studies have been conducted in order to elucidate the mechanism behind the development of MDR cancer cells. One of the most recognized mechanisms involves the ABC (ATP Binding Cassette) transporter proteins. These proteins are capable of coupling the energy of ATP binding and hydrolysis, so as to transport substrates across a cell membrane. The normal physiological role of these proteins is detoxification and clearance by active secretion of intracellular xenobiotic and other undesired substances out of the cell. Thus, in order to ultimately perform their normal physiological role, nature has designed these proteins capable of extruding a wide scope of molecules.

Due to their recognized activity in multidrug resistance (MDR) in tumor chemotherapy these transporter proteins are widely termed in the art as “MDR extrusion pumps”.

The lowered efficacy of chemotherapy is linked to the fact that MDR extrusion pumps are over-expressed in cancer cells, as compared to their expression level in normal cells, and are responsible for pumping chemotherapeutic drugs out of the cell, which reduces the levels of intracellular drug below lethal thresholds regardless of the of nature of the cancer cell and/or the drug.

This mechanism of resistance may account for de novo resistance in common tumors, such as colon cancer and renal cancer, and for acquired resistance, as observed in common hematologic tumors such as acute nonlymphocytic leukemia and malignant lymphomas.

Both the resistance to conventional drugs monotherapy and the toxicity of currently use chemotherapeutic agents, support the rationale for combination drug therapy and the use of agents that can fight MDR. Compounds capable of inhibiting MDR extrusion pumps are known in the art as chemosensitizers or chemosensitizing agents. Combination of drugs with different modes of action may protect normal cells against chemotoxicity [Carvajal, D. et al., Cancer Res., 2005, 65, 1918-1924] or facilitate chemotherapy action on resistant tumors [Molnar, J. et al., Curr. Pharm. Des, 2006, 12, 287-311].

In some embodiments, the conjugates presented herein is designed to carry a variety of anti-cancer drugs that differ from one another in their mechanism of action. This differential mechanism of action can overcome MDR by simultaneously attacking more than one biological system of the malignant cell, causing death before the cell can respond to the attack by the MDR mechanisms.

In the context of some embodiments of the present invention, the term “cancer” refers, but not limited to acute lymphoblastic, acute lymphoblastic leukemia, acute lymphocytic leukemia, acute myelogenous leukemia, acute myeloid leukemia, adrenocortical carcinoma, AIDS-related lymphoma, anal cancer, appendix cancer, basal-cell carcinoma, bladder cancer, brain cancer, brainstem glioma, breast cancer, bronchial adenomas/carcinoids, Burkitt's lymphoma, carcinoid tumor, cerebellar or cerebral astrocytoma, cervical cancer, cholangiocarcinoma, chondrosarcoma, chronic lymphocytic or chronic lymphocytic leukemia, chronic myelogenous or chronic myeloid leukemia, chronic myeloproliferative disorders, colon cancer, cutaneous T-cell lymphoma, desmoplastic small round cell tumor, endometrial uterine cancer, ependymoma, esophageal cancer, Ewing's sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal tumor (GIST), gestational trophoblastic tumor, glioma of the brain stem, hairy cell leukemia, head and neck cancer, heart cancer, hepatocellular (liver) cancer, Hodgkin lymphoma, hypopharyngeal cancer, hypothalamic and visual pathway glioma, intraocular melanoma, Islet cell carcinoma, Kaposi sarcoma, laryngeal cancer, leukaemia, lip and oral cavity cancer, liposarcoma, lymphoma, male breast cancer, malignant mesothelioma, medulloblastoma, melanoma, Merkel cell skin carcinoma, mesothelioma, metastatic squamous neck cancer, mouth cancer, multiple endocrine neoplasia syndrome, multiple myeloma, multiple myeloma/plasma cell neoplasm, mycosis fungoides, myelodysplastic/myeloproliferative diseases, nasal cavity and paranasal sinus cancer, nasopharyngeal carcinoma, neuroblastoma, non-Hodgkin lymphoma, non-melanoma skin cancer, non-small cell lung cancer, oligodendroglioma, oral cancer, oropharyngeal cancer, osteosarcoma and malignant fibrous histiocytoma, ovarian cancer, ovarian germ cell tumor, ovarian epithelial cancer (surface epithelial-stromal tumor), ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonary blastoma, primary carcinoma, primary central nervous system lymphoma, primary liver cancer, prostate cancer, rectal cancer, renal cell carcinoma, renal pelvis and ureter carcinoma, retinoblastoma, rhabdomyosarcoma, salivary gland cancer, Sézary syndrome, small cell lung cancer, small intestine cancer, soft tissue sarcoma, squamous cell carcinoma, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine sarcoma, vaginal cancer, visual pathway and hypothalamic glioma, vulvar cancer, Waldenström macroglobulinemia and Wilms tumor.

### General definitions

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the terms “amine” or “amino”, encompasses primary, secondary and tertiary amines, and describe both a —NR′R″ end group and a —NR′— linking moiety, wherein R′ and R″ are each independently hydrogen, alkyl, cycloalkyl, aryl, as these terms are defined hereinbelow. The amine can be a primary, secondary or tertiary amine. An “ammonium” group refers to a quaternary amine, exhibiting a positive charge, namely a quaternary ammonium cation.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydrogen, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

The term “alkyl” describes a saturated aliphatic hydrocarbon including straight chain (unbranched) and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain. When an alkyl is a linking moiety, it is also referred to herein as “alkylene”, e.g., methylene, ethylene, propylene, etc.

The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. Alternatively, “aryl” as used herein, means a C₆-C₁₄ mono- or poly-cyclic aromatic ring system. Exemplary aryl groups include phenyl, naphthyl, anthryl, phenanthryl, and biphenyl groups. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof. Preferably, the aryl is phenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. The term “heteroaryl” describes a group or part of a group denotes an aromatic monocyclic or bicyclic moiety of 5 to 10 ring atoms in which one or more, preferably one, two, or three, of the ring atom(s) is(are) selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, pyrazolyl, and the like. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof. Representative examples are pyridine, pyrrole, oxazole, indole, purine and the like.

The term “alkaryl” describes an alkyl, as defined herein, which is substituted by one or more aryl or heteroaryl groups. An example of alkaryl is benzyl.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is an end group, as this phrase is defined hereinabove.

As used herein, the term “acyl” refers to a group having the general formula —C(═O)R′, —C(═O)OR′, —C(═O)—O—C(═O)R′, —C(═O)SR′, —C(═O)N(R′)₂, —C(═S)R′, —C(═S)N(R′)₂, and —C(═S)S(R′), —C(═NR′)R″, —C(═NR′)OR″, —C(═NR′)SR″, and —C(═NR′)N(R″)₂, wherein R′ and R″ are each independently hydrogen, halo, substituted or unsubstituted hydroxyl, substituted or unsubstituted thiol, substituted or unsubstituted amine, substituted or unsubstituted acyl, cyclic or acyclic, substituted or unsubstituted, branched or unbranched aliphatic, cyclic or acyclic, substituted or unsubstituted, branched or unbranched heteroaliphatic, cyclic or acyclic, substituted or unsubstituted, branched or unbranched alkyl, cyclic or acyclic, substituted or unsubstituted, branched or unbranched alkenyl, substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

The term “halo” describes fluorine, chlorine, bromine or iodine substituent.

The term “halide” describes an anion of a halogen atom, namely F—, Cl—Br and I—.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O-linking moiety, as these phrases are defined hereinabove, where R′ is as defined hereinabove.

The term “sulfite” describes an —O—S(═O)—O—R′ end group or a —O—S(═O)—O— group linking moiety, as these phrases are defined hereinabove, where R′ is as defined hereinabove.

The term “thiosulfite” describes a —O—S(═S)—O—R′ end group or an —O—S(═S)—O— group linking moiety, as these phrases are defined hereinabove, where R′ is as defined hereinabove.

The term “sulfinate” or “sulfinyl” describes a —S(═O)—OR′ end group or an —S(═O)—O— group linking moiety, as these phrases are defined hereinabove, where R′ is as defined hereinabove.

The terms “solfoxide” or “sulfinyl” describe a —S(═O)R′ end group or an —S(═O)— linking moiety, as these phrases are defined hereinabove, where R′ is as defined hereinabove.

The term “sulfonate” or “sulfonyl” describes a —S(═O)₂—R′ end group or an —S(═O)₂-linking moiety, as these phrases are defined hereinabove, where R′ is as defined herein. Alternatively, “sulfonyl” means a —SO₂R radical, where R s alkyl, haloalkyl, aryl, aralkyl, heteroaryl, heteroaralkyl, heterocyclyl, heterocyclylalkyl, each as defined herein and wherein the aryl, heteroaryl, or heterocyclyl ring either alone or part of another group.

The term “S-sulfonamide” describes a —S(═O)₂—NR′R″ end group or a —S(═O)₂—NR′-linking moiety, as these phrases are defined hereinabove, with R′ and R″ as defined herein.

The term “N-sulfonamide” describes an R′S(═O)₂—NR″— end group or a —S(═O)₂—NR′-linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphate” describes an —O—P(═O)₂(OR′) end or reactive group or a —O—P(═O)₂(O)— linking moiety, as these phrases are defined hereinabove, with R′ as defined herein.

The term “phosphonate” describes a —P(═O)(OR′)(OR″) end or reactive group or a —P(═O)(OR′)(O)— linking moiety, as these phrases are defined hereinabove, with R′ and R″ as defined herein.

The term “thiophosphonate” describes a —P(═S)(OR′)(OR″) end group or a —P(═S)(OR′)(O)— linking moiety, as these phrases are defined hereinabove, with R′ and R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)-linking moiety, as these phrases are defined hereinabove, with R′ as defined herein.

The term “oxo” as used herein, described a ═O end group.

The term “thioxo” as used herein, described a ═S end group.

The term “oxime” describes a ═N—OH end group or a ═N—O— linking moiety, as these phrases are defined hereinabove.

The term “hydroxyl” describes a —OH group.

As used herein, the term “aldehyde” refers to an —C(═O)—H group.

The term “acyl halide” describes a —(C═O)R″″ group wherein R″″ is halo, as defined hereinabove.

The term “alkoxy” as used herein describes an —O-alkyl, an —O-cycloalkyl, as defined hereinabove. The ether group —O— is also a possible linking moiety.

The term “aryloxy” describes both an —O-aryl and an —O-heteroaryl group, as defined herein.

The term “disulfide” as used herein describes an —S—S— linking moiety, which in some cases forms between two thiohydroxyl groups.

The terms “thio”, “sulfhydryl” or “thiohydroxyl” as used herein describe an —SH group.

The term “thioalkoxy” or “thioether” describes both a —S-alkyl group, and a —S-cycloalkyl group, as defined herein. The thioether group —S— is also a possible linking moiety.

The term “thioaryloxy” describes both a —S-aryl and a —S-heteroaryl group, as defined herein. The thioarylether group —S-aryl- is also a possible linking moiety.

The term “cyano” or “nitrile” describes a —C≡N group.

The term “isocyanate” describes an —N═C═O group.

The term “nitro” describes an —NO₂ group.

The term “alkylsilyl”, as used herein, encompasses —R′—Si(R″)₃ end group or a —R—Si(R′)₂— linking moiety, as these phrases are defined hereinabove, where R′ and R″ is as defined herein.

The term “carboxylate” or “ester”, as used herein encompasses C-carboxylate and O-carboxylate.

The term “C-carboxylate” describes a —C(═O)—OR′ end group or a —C(═O)—O— linking moiety, as these phrases are defined hereinabove, where R′ is as defined herein.

The term “O-carboxylate” describes a —OC(═O)R′ end group or a —OC(═O)— linking moiety, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O— linking moiety, as these phrases are defined hereinabove, where R′ is as defined herein.

The term “O-thiocarboxylate” describes a —OC(═S)R′ end group or a —OC(═S)— linking moiety, as these phrases are defined hereinabove, where R′ is as defined herein.

The term “carbamate” as used herein encompasses N-carbamate and O-carbamate.

The term “N-carbamate” describes an R″OC(═O)—NR′— end group or a —OC(═O)—NR′-linking moiety, as these phrases are defined hereinabove, with R′ and R″ as defined herein.

The term “O-carbamate” describes an —OC(═O)—NR′R″ end group or an —OC(═O)—NR′— linking moiety, as these phrases are defined hereinabove, with R′ and R” as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking moiety, as these phrases are defined hereinabove, with R′ and R″ as defined herein.

The term “N-thiocarbamate” describes an R″OC(═S)NR′— end group or a —OC(═S)NR′-linking moiety, as these phrases are defined hereinabove, with R′ and R″ as defined herein.

The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′-linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking moiety, with R′, R″ and R′″ as defined herein.

The term “amide” as used herein encompasses C-amide and N-amide.

The term “C-amide” describes a —C(═O)—NR′R″ end group or a —C(═O)—NR′— linking moiety, as these phrases are defined hereinabove, where R′ and R″ are as defined herein.

The term “N-amide” describes a R′C(═O)—NR″— end group or a R′C(═O)—N— linking moiety, as these phrases are defined hereinabove, where R′ and R″ are as defined herein.

The term “imine”, which is also referred to in the art interchangeably as “Schiff-base”, describes a —N═CR′— linking moiety, with R′ as defined herein or hydrogen. As is well known in the art, Schiff bases are typically formed by reacting an aldehyde or a ketone and an amine-containing moiety such as amine, hydrazine, hydrazide and the like, as these terms are defined herein. The term “aldimine” refers to a —CH═N— imine which is derived from an aldehyde. The term “ketimine” refers to a —CR′═N— imine which is derived from a ketone.

The term “hydrazone” refers to a —R′C═N—NR″— linking moiety, wherein R′ and R″ are as defined herein.

The term “semicarbazone” refers to a linking moiety which forms in a condensation reaction between an aldehyde or ketone and semicarbazide. A semicarbazone linking moiety stemming from a ketone is a —R′C═NNR″C(═O)NR′″—, and a linking moiety stemming from an aldehyde is a —CR′═NNR″C(═O)NR′″—, wherein R′ and R″ are as defined herein and R′″ or as defined for R′.

As used herein, the term “lactone” refers to a cyclic ester, namely the intra-condensation product of an alcohol group —OH and a carboxylic acid group —COOH in the same molecule.

As used herein, the term “lactam” refers to a cyclic amide, as this term is defined herein. A lactam with two carbon atoms beside the carbonyl and four ring atoms in total is referred to as a β-lactam, a lactam with three carbon atoms beside the carbonyl and five ring atoms in total is referred to as a γ-lactam, a lactam with four carbon atoms beside the carbonyl and six ring atoms in total is referred to as a δ-lactam, and so on.

The term “guanyl” describes a R′R″NC(═N)— end group or a —R′NC(═N)— linking moiety, as these phrases are defined hereinabove, where R′ and R″ are as defined herein.

The term “guanidine” describes a —R′NC(═N)—NR″R′″ end group or a —R′NC(═N)—NR″— linking moiety, as these phrases are defined hereinabove, where R′, R″ and R′″ are as defined herein.

The term “hydrazine” describes a —NR′—NR″R′″ end group or a —NR′—NR″— linking moiety, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking moiety, as these phrases are defined hereinabove, where R′, R″ and R′″ are as defined herein.

The term “hydroxylamine”, as used herein, refers to either a —NHOH group or a —ONH₂.

As used herein, the terms “azo” or “diazo” describe a —N═N—R′ end group or a —N═N-linking moiety, as these phrases are defined hereinabove, where R′ is as defined herein.

As used herein, the term “azido” described a —N═N+=N—(—N₃) end group.

The term “triazine” refers to a heterocyclic ring, analogous to the six-membered benzene ring but with three carbons replaced by nitrogen atoms. The three isomers of triazine are distinguished from each other by the positions of their nitrogen atoms, and are referred to as 1,2,3-triazine, 1,2,4-triazine, and 1,3,5-triazine. Other aromatic nitrogen heterocycles include pyridines with 1 ring nitrogen atom, diazines with 2 nitrogen atoms in the ring and tetrazines with 4 ring nitrogen atoms.

The term “triazole” refers to either one of a pair of isomeric chemical compounds with molecular formula C₂H₃N₃, having a five-membered ring of two carbon atoms and three nitrogen atoms, namely 1,2,3-triazoles and 1,2,4-triazoles.

The term “aziridine”, as used herein, refers to a reactive group which is a three membered heterocycle with one amine group and two methylene groups, having a molecular formula of —C₂H₃NH.

As used herein, the term “thiohydrazide” describes a —C(═S)—NR′—NR″R′″ end group or a —C(═S)—NR′—NR″— linking moiety, as these phrases are defined hereinabove, where R′, R″ and R′″ are as defined herein.

As used herein, the term “methyleneamine” describes an —NR′—CH₂—CH═CR″R′″ end group or a —NR′—CH₂—CH═CR″— linking moiety, as these phrases are defined hereinabove, where R′, R″ and R′″ are as defined herein.

The term “diene”, as used herein, refers to a —CR′═CR″—CR′″ ═CR″″— group, wherein R′ as defined hereinabove, and R″, R′″ and R″″ are as defined for R′.

The term “dienophile”, as used herein, refers to a reactive group that reacts with a diene, typically in a Diels-Alder reaction mechanism, hence a dienophile is typically a double bond or an alkenyl.

The term “epoxy”, as used herein, refers to a reactive group which is a three membered heterocycle with one oxygen and two methylene groups, having a molecular formula of —C₂H₃O.

The phrase “covalent bond”, as used herein, refers to one or more pairs of electrons that are shared between atoms in a form of chemical bonding.

It is expected that during the life of a patent maturing from this application many relevant anticancer conjugates will be developed and the scope of the phrase “anticancer conjugate” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### General Synthesis

Scheme 1 presents a general synthetic strategy for affording the conjugates provided herein, wherein drugs were conjugated to the protected carbamate form of Azene. The Azene compound was prepared following the below scheme:

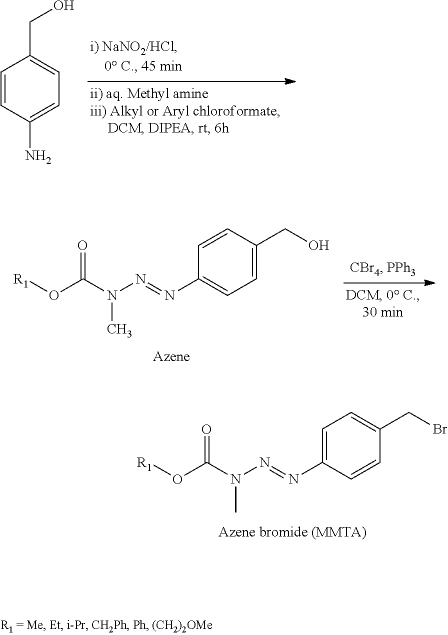

Methyl triazene tether Azene through self-emulative 4-aminobenzyl linker to yield corresponding chimeras shown in Scheme 1. The main requirement for the drug to be chimerical is the existence of a free functional group such as amine, hydroxy and thiol for alkylation reaction with the Azene bromide.

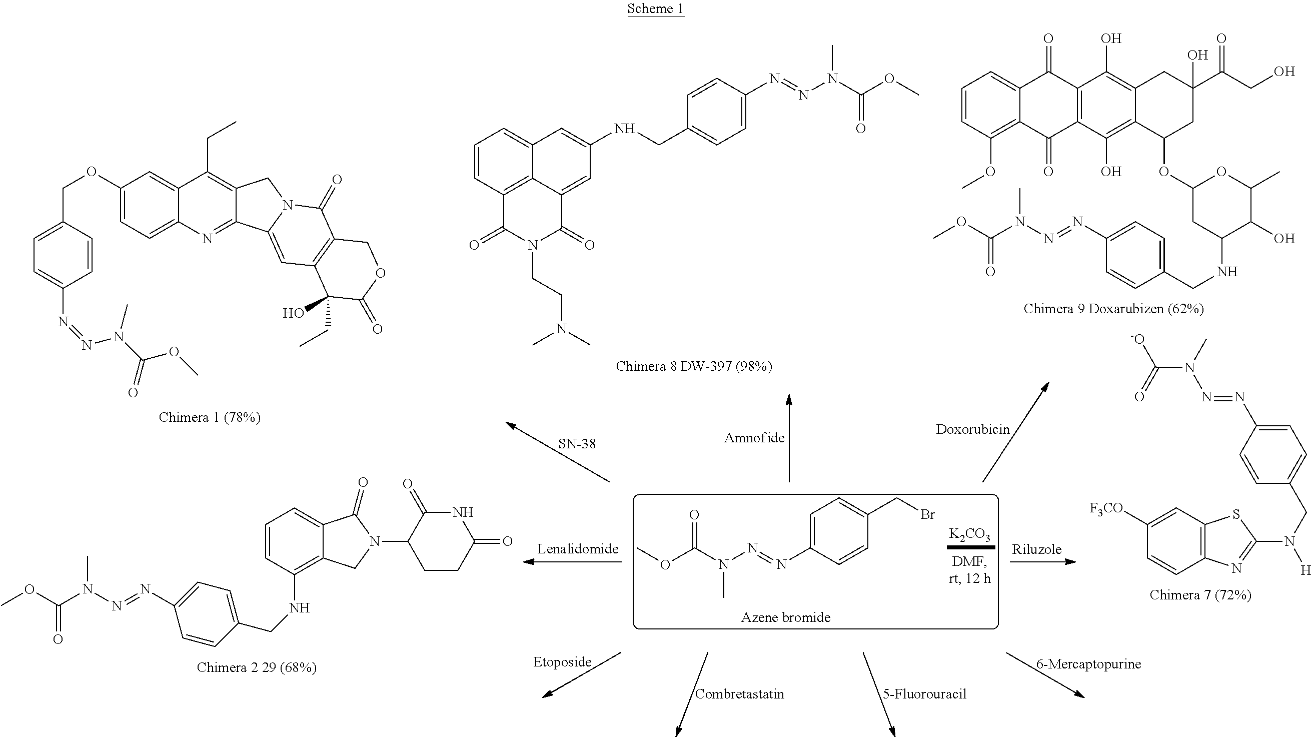

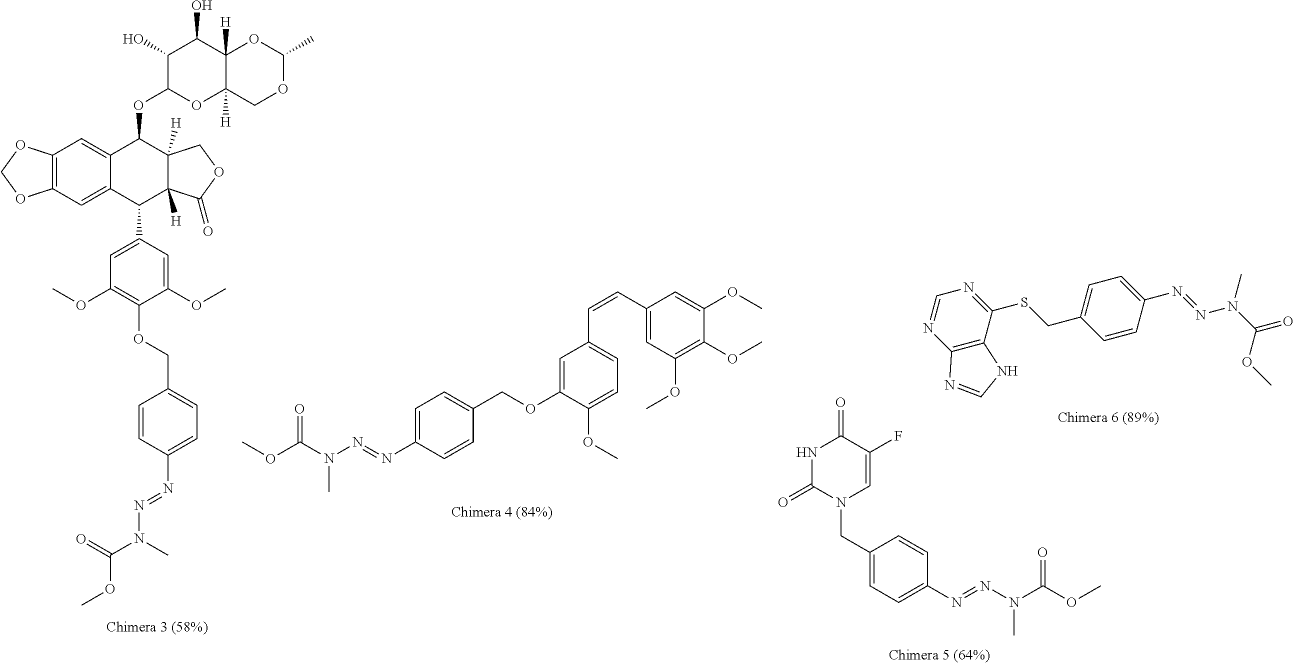

It was found that general reaction conditions for alkylation of all above mentioned drugs, namely 1 equiv. of Azene bromide, 1.2 equiv. of K₂CO₃ in DMF at room temperature for 12 h under N₂, were satisfactory for yielding relatively clean crude products. Pure products were obtained after flash chromatography on silica using ethyl acetate/petroleum ether or DCM/MeOH gradients in good yields (58%-98%) and their structures confirmed by ¹H&¹³C NMR and HRMS.

### Synthesis of Anthracycline Chimeras

The Azene compound was used in the following conjugation reaction, with which some of the conjugates provided herein were prepared:

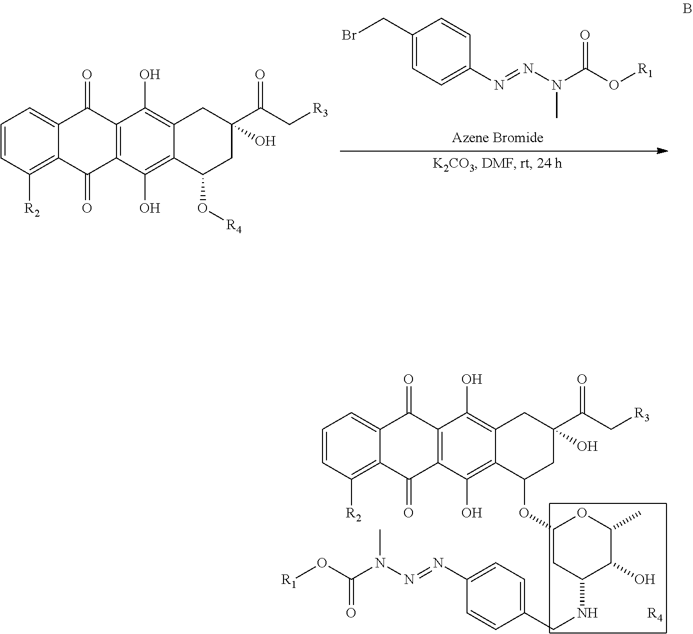

and the resulting conjugates are:

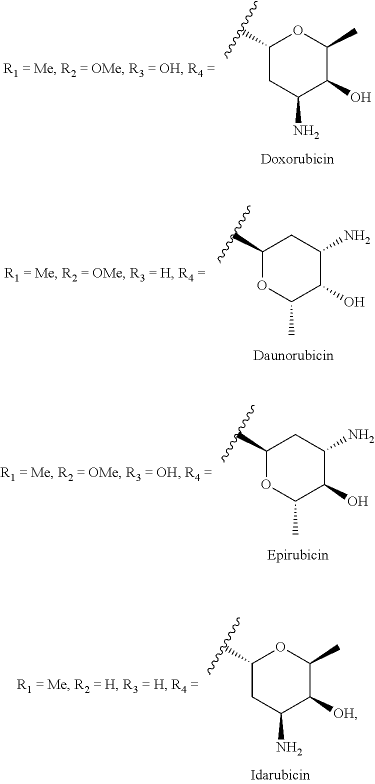

namely:

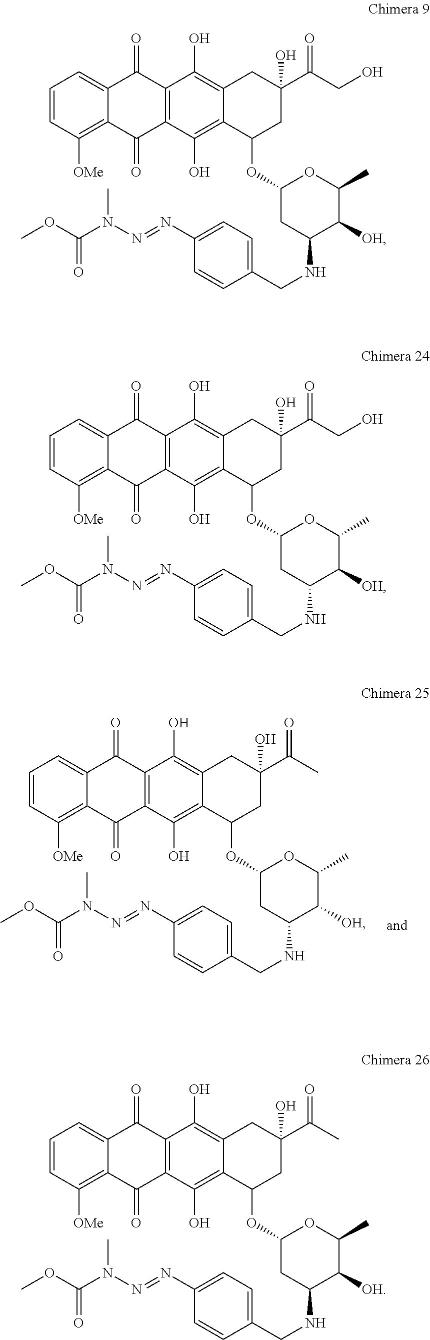 [SMILES: COC(=O)N(C)/N=N/c1ccc(CN[C@H]2C[C@H](OC3C[C@](O)(C(=O)CO)Cc4c(O)c5c(c(O)c43)C(=O)c3c(OC)cccc3C5=O)O[C@@H](C)[C@H]2O)cc1 ; COC(=O)N(C)/N=N/c1ccc(CN[C@@H]2C[C@H](OC3C[C@](O)(C(=O)CO)Cc4c(O)c5c(c(O)c43)C(=O)c3c(OC)cccc3C5=O)O[C@H](C)[C@H]2O)cc1 ; COC(=O)N(C)/N=N/c1ccc(CN[C@@H]2C[C@H](OC3C[C@](O)(C(C)=O)Cc4c(O)c5c(c(O)c43)C(=O)c3c(OC)cccc3C5=O)O[C@H](C)[C@@H]2O)cc1 ; "and" ; COC(=O)N(C)/N=N/c1ccc(CN[C@H]2C[C@H](OC3C[C@](O)(C(C)=O)Cc4c(O)c5c(c(O)c43)C(=O)c3c(OC)cccc3C5=O)O[C@@H](C)[C@H]2O)cc1]

### Example 2

### Chemistry Material and Methods

Drugs and other reagents were purchased from Tzamal D-Chem Laboratories Ltd. Petah-Tikva, Israel. Chemical reactions were monitored by TLC (Silica gel 60 F-254, Merck). ¹H and ¹³C NMR spectra were measured by using a 400 MHz Bruker Avance III HD (¹H 400 MHz and ¹³C 100 MHz) spectrometer in CD30D and DMSO-d6. All the solvents were from Bio-Lab Ltd. Jerusalem, Israel or Gas Technologies Ltd. Kefar Saba, Israel. All other chemicals were from Holland Moran or Sigma-Aldrich. HRMS was performed in ESI positive mode by using an Agilent 6550 iFunnel Q-TOF LC-MS instrument. IR spectra were recorded as KBr tablets on a Bruker FTIR ALPHA II spectrometer equipped with a platinum diamond attenuated total reflectance (ATR) module. Purification of the compounds was done via normal phase chromatography by using silica gel (Silica flash P60) supplied by Merck. The column was kept at room temperature. The eluents were petroleum ether, ethyl acetate, chloroform and methanol. Electron spray mass spectra (ESI-MS) were obtained using an Autoflex III smart-beam (MALDI, Bruker), Q-TOF micro (Waters) or an LCQ Fleet™ ion trap mass spectrometer (Finnigan/Thermo). HPLC/LC-MS analyses were made using Agilent infinity 1260 connected to Agilent quadruple LC-MS 6120 series equipped with ZORBAX SB—C18, 2.1×50 mm, 1.8 mm column. In all cases, the eluent was A (0.1% FA in H₂O) and B (0.1% FA in ACN) and the elution gradient profile was: 100% A for first 3 min, followed by 5 min (from min 3 to min 8) during which it reached 100% B, followed by 5 min (from min 8 to min 13) of 100% B, followed by 2 min (from min 13 to min 15) during which it returned back to A, followed by 2 min (from min 15 to min 17) of 100% A. The UV detection was at 254 nm. The column temperature was kept at 50° C. The flow rate was of 0.3 mL/min. The MS fragmentor was tuned on 30 V or 70 V on positive or negative mode.

### Example 3

### Synthesis Procedure

The drug (50 mg, 1 equiv.) was dissolved in dry DMF (5 mL) under a nitrogen atmosphere and the reaction mixture cooled down to 0° C. K₂CO₃ (3 equiv.) was added and the reaction mixture was stirred at 0° C. for 15 minutes. Then, Azene bromide [²⁵](1.2 equiv.) was added dropwise to the mixture, and the solution was stirred at room temperature for 12 h. After completion of the reaction (monitoring by TLC), the reaction mixture was quenched with water (10 mL) and extracted with CH₂Cl₂ (2×20 mL). The organic phase was dried over Na₂SO₄ and then filtered. Solvent was removed using a rotary evaporator, and the crude material was purified by silica gel chromatography.

Doxorubizen or (E)-methyl-3-(4-(((3-hydroxy-2-methyl-6-((3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)amino)methyl)phenyl)-1-methyltriaz-2-ene-1-carboxylate (also referred to herein as Chimera 9):

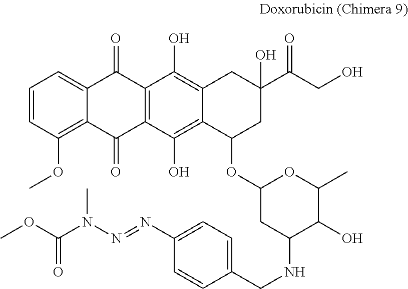

Doxorubicin (Chimera 9) was synthesized using doxorubicin (50 mg) and purified by silica gel column chromatography.[MeOH:DCM (2:8), v/v]; yield: 42 mg (62%), R_{f}=0.66, Red Solid; ¹H NMR (400 MHz, CDCl₃): δ (ppm) 8.02 (d, J=7.6 Hz, 1H), 7.78 (t, J=8.1 Hz, 1H), 7.52 (d, J=8.1 Hz, H), 7.39 (d, J=8.3 Hz, 1H), 7.31 (d, J=8.2 Hz, 2H), 5.53 (br. s., 1H), 5.31 (br. s., 1H), 4.75 (s, 2H), 4.69 (br. s., 1H), 4.08 (s, 3H), 3.97 (m, 1H), 3.95 (s, 3H), 3.85 (d, J=13.2 Hz, 1H), 3.76 (d, J=13.1 Hz, 1H), 3.68 (br. s., 1H), 3.43 (s, 3H), 3.25 (m, 1H), 3.03-2.94 (m, 2H), 2.36 (d, J=14.8 Hz, 1H), 2.15 (d, J=14.5 Hz, 1H), 1.85 (m, 1H), 1.71 (d, J=13.4 Hz, 1H), 1.37 (d, J=6.5 Hz, 3H); ¹³C NMR (101 MHz, CDCl₃): δ (ppm) 213.9, 187.2, 186.9, 161.2, 156.3, 155.8, 155.1, 148.3, 135.9, 135.6, 133.9, 133.7, 129.0, 122.5, 121.0, 120.0, 118.6, 111.7, 100.9, 69.5, 67.0, 66.9, 65.6, 56.8, 54.2, 52.6, 49.9, 35.6, 34.1, 30.4, 30.1, 17.3. HRMS (ESI) m/z calcd for C₃₇H₄₀N₄O₁₃ [M+H]+: 749.2670, found: 749.2670.

(S,Z)-methyl3-(4-(((4,11-diethyl-4-hydroxy-3,14-dioxo-3,4,12,14-tetrahydro-1H pyrano [3′,4′:6,7]indolizino[1,2-b]quinolin-9-yl)oxy)methyl)phenyl)-1-methyltriaz-2-ene-1-carboxylate (also referred to herein as Chimera 1): The compound was synthesized from SN—38 (50 mg) and purified by silica gel column chromatography [MeOH:DCM (1:9), v/v]; yield: 59 mg (78%), R_{f}=0.74, Brown solid; ¹H NMR (400 MHz, CDCl₃) δ=8.13 (d, J=9.2 Hz, 1H), 7.67 (s, 3H), 7.60 (s, 4H), 7.64 (s, 3H), 7.54 (d, J=8.3 Hz, 7H), 7.48 (dd, J=2.6, 9.3 Hz, 4H), 7.30 (d, J=2.4 Hz, 3H), 5.70 (d, J=16.1 Hz, 3H), 5.28 (s, 3H), 5.23 (s, 2H), 5.15 (s, 2H), 4.19 (br. s., 1H), 3.97 (s, 3H), 3.96 (d, 1H) 3.47 (s, 3H), 3.44 (d, 1H), 3.06 (d, J=7.8 Hz, 2H), 1.88 (dd, J=7.5, 9.3 Hz, 2H), 1.32 (t, J=7.6 Hz, 3H), 0.99 (t, J=7.4 Hz, 3H); ¹³C NMR (101 MHz, CDCl₃) δ=174.0, 157.8, 157.7, 155.0, 150.4, 149.8, 148.9, 147.3, 145.5, 143.8, 136.9, 132.3, 128.3, 128.1, 127.4, 122.8, 122.6, 118.0, 103.4, 97.6, 73.0, 70.1, 66.4, 54.3, 49.5, 31.7, 30.4, 23.2, 13.6, 7.9 ppm. HRMS (ESI) m/z calcd for C₃₂H₃₁N₅O₇ [M+H]+: 598.2301, found: 598.2302.

(E)-methyl 3-(4-(((2-(2,6-dioxopiperidin-3-yl)-1-oxoisoindolin-4-yl)amino) methyl)phenyl)-1-methyltriaz-2-ene-1-carboxylate (also referred to herein as Chimera 2): The compound was synthesized from lenalidomide (50 mg) and purified by silica gel column chromatography.[ethyl acetate:petroleum ether (6:4), v/v]; yield: 61 mg (68%), R_{f}=0.53, white solid; ¹H NMR (400 MHz, CDCl₃) δ=7.50 (d, J=8.3 Hz, 2H), 7.40 (d, J=8.3 Hz, 2H), 7.30 (br. s., 1H), 7.27-7.21 (m, 1H), 6.82 (d, J=7.5 Hz, 1H), 5.19 (dd, J=5.1, 13.3 Hz, 1H), 5.00-4.85 (m, 2H), 4.21 (s, 1H), 4.12-4.09 (s, 1H), 3.95 (s, 3H), 3.44 (s, 3H), 2.95 (m, 1H), 2.90-2.76 (m, 1H), 2.30-2.15 (m, 1H), 2.10 (d, J=5.1 Hz, 1H); ¹³C NMR (101 MHz, CDCl₃) δ=171.1, 170.2, 169.9, 155.0, 148.4, 141.4, 137.6, 132.4, 129.8, 129.6, 126.3, 122.3, 118.2, 114.4, 54.2, 52.7, 45.1, 43.4, 32.2, 30.4, 22.7 ppm. HRMS (ESI) m/z calcd for C₂₃H₂₄N₆O₅ [M+Na]+487.1706, found: 487.1709.

(E)-methyl3-(4-((4-((5R,5aR,8aR,9S)-9-(((2R,4aR,7R,8R,8aS)-7,8-dihydroxy-2-(methylhexahydropyrano[3,2-d][1,3]dioxin-6-yl)oxy)-6-oxo-5,5a,6,8,8a,9-hexahydrofuro [3′,4′:6,7]naphtho[2,3-d][1,3]dioxol-5-yl)-2,6-dimethoxyphenoxy) methyl)phenyl)-1-methyltriaz-2-ene-1-carboxylate (also referred to herein as Chimera 3): The compound was synthesized from Etoposide (50 mg) and purified by silica gel column chromatography [ethyl acetate:petroleum ether (5:5), v/v]; yield: 39 mg (58.20%), R_{f}=0.61, white Solid; ¹H NMR (400 MHz, CDCl₃) δ=7.64 (d, J=8.3 Hz, 2H), 7.55 (d, J=8.3 Hz, 2H), 6.80 (s, 1H), 6.44 (s, 2H), 6.24 (s, 1H), 6.01-5.92 (s, 2H), 5.06 (s, 2H), 4.93 (d, J=3.1 Hz, 1H), 4.72 (d, J=5.0 Hz, 1H), 4.65-4.58 (m, 1H), 4.56-4.37 (m, 2H), 4.25 (d, J=4.9 Hz, 1H), 4.16 (dd, J=5.0, 10.3 Hz, 2H), 3.98 (s, 73H), 3.96 (d, J=7.6 Hz, 1H), 3.79 (s, 6H), 3.71 (s, 3H), 3.62-3.53 (m, 2H), 3.48 (m, 3H), 3.46-3.41 (m, 2H), 3.35-3.26 (m, 2H), 3.22 (dd, J=5.0, 9.8 Hz, 1H), 3.15 (dd, J=4.8, 9.7 Hz, 1H), 3.02-2.95 (m, 1H), 2.90-2.85 (m, 1H), 1.36 (d, J=5.0 Hz, 3H)¹³C NMR (101 MHz, CDCl₃) δ (ppm) 180.5, 155.2, 153.9, 152.9, 148.7, 146.5, 138.9, 136.0, 133.2, 129.2, 126.2, 122.1, 109.8, 108.6, 106.1, 101.5, 99.8, 79.9, 75.5, 74.6, 74.6, 73.2, 69.6, 66.6, 56.4, 54.2, 44.4, 44.2, 39.6, 30.3, 20.4; HRMS (ESI) m/z calcd for C₃₉H₄₃N₃O₁₅ [M+H]+: 794.2772, found: 794.2769; or C₃₉H₄₃N₃O₁₅ [M+Na]+: 816.2592, found: 816.2594.

(E)-methyl3-(4-((2-methoxy-5-((Z)-3,4,5-trimethoxystyryl) phenoxy)methyl) phenyl)-1-methyltriaz-2-ene-1-carboxylate (also referred to herein as Chimera 4): The compound was synthesized from combretastatin (50 mg) and purified by silica gel column chromatography [ethyl acetate:petroleum ether (2:8), v/v]; yield: 69 mg (84%), R_{f}=0.65, white solid; ¹H NMR (400 MHz, CDCl₃) δ=7.58 (d, J=8.6 Hz, 2H), 7.37 (d, J=8.6 Hz, 2H), 6.86 (d, J=12 Hz, 1H), 6.8 (d, J=12 Hz, 1H), 6.50 (s, 2H), 6.44 (d, J=1.6 Hz, 2H), 4.97 (s, 2H), 3.98 (s, 3H), 3.88 (s, 3H), 3.85 (s, 3H), 3.71 (s, 3H), 3.48 (s, 3H); ¹³C NMR (101 MHz, CDCl₃) δ=155.1, 153.1, 149.1, 148.5, 147.6, 137.9, 137.2, 133.2, 129.9, 129.7, 129.0, 128.0, 122.8, 122.4, 114.7, 111.6, 106.1, 70.6, 61.1, 56.1, 56.1, 54.2, 30.7 ppm. HRMS (ESI) m/z calcd for C₂₈H₃₁N₃O₇ [M+Na]+: 544.2060, found: 544.2065.

(E)-methyl3-(4-((5-fluoro-2,4-dioxo-3,4-dihydropyrimidin-1(2H)-yl)methyl) phenyl)-1-methyltriaz-2-ene-1-carboxylate (also referred to herein as Chimera 5): The compound was synthesized from 5-fluorouracil (50 mg) and purified by silica gel column chromatography. [ethyl acetate:petroleum ether (4:6), v/v]; yield: 82 mg (64%), R_{f}=0.71, white Solid; ¹H NMR (400 MHz, CDCl₃) δ=7.63 (d, J=8.44 Hz, 11H)-7.34 (d, J=8.56 Hz, 12H), 7.20 (s, 1H), 4.91 (s, 2H), 3.97 (s, 3H), 3.47 (s, 3H); ¹³C NMR (101 MHz, CDCl₃) δ=155.0, 150.3, 149.3, 148.7, 136.8, 135.1, 130.2, 129.2, 123.1, 122.4, 54.4, 52.2, 30.4; HRMS (ESI) m/z calcd for C₁₄H₁₄FN₅O₄[M+Na]+: 358.0927, found: 358.0930.

(E)-methyl 3-(4-(((7H-purin-6-yl)thio)methyl)phenyl)-1-methyltriaz-2-ene-1-carboxylate (also referred to herein as Chimera 6): The compound was synthesized from 6-mercaptopurine (50 mg) and purified by silica gel column chromatography [ethyl acetate:petroleum ether (5:5), v/v]; yield: 104 mg (89%), R_{f}=0.72, light yellow solid. ¹H NMR (400 MHz, DMSO-d₆): δ (ppm)=8.74 (s, 1H), 8.45 (s, 1H), 7.58 (d, J=8.6 Hz, 2H), 7.49 (d, J=8.4 Hz, 2H), 4.70 (s, 2H), 3.88 (s, 3H), 3.36 (s, 3H); ¹³C NMR (DMSO-d₆, 10|MHz): δ (ppm) 154.1, 151.4, 147.3, 138.9, 130.0, 121.8, 121.8, 54.0, 31.2, 30; HRMS (ESI) m/z calcd for C₁₅H₁₅N₇O₂S [M+Na]+: 380.0906, found: 380.0909.

For (E)-methyl1-methyl-3-(4-(((6-(trifluoromethoxy)benzo [d]thiazol2yl)amino) methyl)phenyl) triaz-2-ene-1-carboxylate (also referred to herein as Chimera 7): The compound was synthesized from Riluzole (50 mg) and purified by silica gel column chromatography [ethyl acetate:petroleum ether (6:4), v/v]; yield: 67 mg (72%), R_{f}=0.61, white Solid; ¹H NMR (400 MHz, CDCl₃) δ (ppm) 7.41 (d, J=8.3 Hz, 2H), 7.25 (d, J=8.3 Hz, 2H) 7.22 (m, 1H), 7.18 (d, J=8.8 Hz, 1H), 6.93 (d, J=7.9 Hz, 1H), 4.46 (s, 2H), 3.78 (s, 3H), 3.28 (s, 3H); ¹³C NMR (101 MHz, CDCl₃) δ (ppm) 168.2, 155.1, 151.0, 148.7, 143.8, 137.9, 131.1, 128.5, 122.7, 119.9, 119.2, 114.2, 54.2, 49.1, 30.4; HRMS (ESI) m/z calcd for C₁₈H₁₆F₃N₅O₃S [M+H]+: 440.1004, found: 440.1004.

(E)-methyl 3-(4-(((2-(2-(dimethylamino)ethyl)-1,3-dioxo-2,3-dihydro-1H-benzo [de]isoquinolin-5-yl)amino)methyl)phenyl)-1-methyltriaz-2-ene-1-carboxylate (also referred to herein as Chimera 8): The compound was synthesized from amonafide (50 mg) and purified by silica gel column chromatography [CH₂Cl₂:MeOH (9:1), v/v]; yield: 73 mg (86%), Rf=0.63, brown solid, mp 178° C.; ¹H NMR (400 MHz, DMSO-d₆) δ (ppm) 8.11 (dd, J=2.0 Hz, 1H), 8.08 (dd, 1H), 8.01 (d, J=2.2 Hz, 1H), 7.76 (d, J=8.4 Hz, 2H), 7.65 (d, J=8.4 Hz, 2H) 7.61-7.63 (m, 1H), 7.33 (d, J=2.3 Hz, 1H), 6.06 (s, 1H), 4.78 (s, 2H), 4.59-4.51 (t, 2H), 3.91 (s, 3H), 3.64-3.56 (t, 3H), 3.41 (s, 3H), 3.17 (s, 6H); ¹³C NMR (101 MHz, DMSO-d₆) δ (ppm) 163.8, 163.6, 154.0, 149.6, 147.9, 134.3, 133.6, 131.9, 128.2, 126.9, 125.6, 122.4, 122.0, 121.8, 121.6, 120.7, 112.0, 66.3, 60.1, 54.2, 49.3, 33.4, 30.46. HRMS (ESI) m/z calcd for C₂₆H₂₈N₆O₄ [M+1]+489.2250, found: 489.2260.

### Example 4

### Biochemical Methods and Assays

### Cell Culture:

The human B-cell leukemia cell lines MUTZ5, MHH-CALL4; T-cell leukemia cell line DND41 and the erythroleukemic HEL cells were kindly provided by Prof. Shai Izraeli, (Schneider Children's Medical Center, Petach Tikvah, Israel). Philadelphia like leukemia cell line BV-173 was kindly provided by Dr. Michael Milyavsky (Tel-Aviv University, Tel-Aviv, Israel). The human mantle cell lymphoma cell lines Jeko-1, Mino and Rec-1 were purchased from ATCC. Breast Cancer cell-line MDA-MB-328 (TNBC) was kindly provided by Prof. Albert Pinhasov (Ariel University, Ariel, Israel). The above cells were cultured in RPMI medium (Gibco, Thermo Fisher Scientific Inc., USA). Granta cells were kindly provided by Prof. Martin Dreyling (University of Munich, Munich, Germany) and were grown in Dulbecco's Modified Eagle Medium (DMEM, Gibco, Thermo Fisher Scientific Inc., USA). OCI-AML3 and KG-1a were kindly provided by Dr. Liran Shlush (Weizmann institute, Rehovot, Israel) and were grown in Iscove's Modified Dulbecco's Medium (IMDM, Gibco, Thermo Fisher Scientific Inc., USA). All media were supplemented with 10% heat-inactivated Fetal Bovine Serum (FBS, Biological industries, Israel), 2 mM glutamine (Biological industries, Israel) and 1% penicillin and streptomycin (Biological industries, Israel). Cells were cultured at 37° C. in a humidified incubator with 5% CO₂.

### In Vitro Cytotoxicity Assay:

Growth inhibition in hematological malignancies cell-lines was measured by tetrazolium WST-1 assay (Roche, Basel, Switzerland). Cells (1*10⁴) were treated with various concentrations of the specific chimera or parental drug. After 48 h of incubation, 10 μl of WST-1 reagent was added to the plate and incubated for 1 h at 37° C., at which point the absorbance (450 nm) was measured in an ELISA plate reader (Synergy HTX multi-mode reader, Winooski, Vermont, USA).

The cytotoxicity of the synthesized chimeras on MDA MB-328 was determined by measuring the mitochondrial enzyme activity, using a commercial XTT assay kit (Biological Industries, Bet-Ha'emek, Israel). All samples contained DMSO (Sigma Aldrich) at final concentration of the second incubation, XTT reagent was added, and the cells were re-incubated for additional 3 h. During that time, the absorbencies in the wells were measured with a TECAN Infinite M200 ELISA reader at both 480 and 680 nm. The difference between these measurements was used for calculating the % Growth Inhibition (GI) in test wells compared to two controls: cells that were exposed to the medium and solvent, and those which were exposed to a solvent-free medium. All the tests were done in tetra-plicate; each experiment was conducted three times.

### Mitochondrial Depolarization:

Changes in mitochondrial potential and cellular plasma membrane permeabilization, were determined with the Muse® Mitochondrial Kit (Luminex, USA). Briefly, Cells were treated with DMSO or the relevant chimera for 48 hours. The cells were incubated with MitoPotential working solution for 20 minutes at 37° C. in 5% CO2. Then, 5 μL Muse® MitoPotential 7-ADD reagent were added and the cells were incubated for 5 minutes at room temperature. The percentage of four cell populations: live (mitopotential+/7−AAD−), depolarized live (mitopotential−/7−AAD−), dead (mitopotential+/7−AAD+), and depolarized dead (mitopotential−/7−AAD+) were measured using the Muse® Cell Analyzer (Luminex, USA).

### Apoptosis Assay:

Cells were analyzed using the multifunctional Muse®AnnexinV and Dead Cell kit (Luminex/Millipore, Austin, Texas, USA). Cells were treated with indicated concentrations of Doxorubizen, the corresponding parent drug, or with DMSO. After 48 hours of incubation cells were harvested, washed twice in PBS, and 2×10⁵ cells were stained with 100 μl of Muse AnnexinV and Dead Cell Reagent. Samples were incubated for 20 min at room temperature in the dark. Cells were analyzed by the Muse®CellAnalyzer system (Millipore, Billerica, MA, USA), and the percentage of apoptosis was determined using Guava software (Luminex/Millipore, USA, version 3.3).

DNA Damage by Dual Staining of pH2A.X and pATM:

BV-173 cells (2×10⁵ cells/well) were treated with DMSO, Doxorubicin or Doxorubizen. After incubation for 3 hours, phosphorylation level of ATM and H2AX was measured by Muse cell analyzer using Muse Multi-Color DNA Damage Kit (Luminex Corporation, Part number: MCH200107) according to manufacturer's instructions. Phosphorylation levels were determined using Guava software (Luminex/Millipore, USA, version 3.3).

### MDA-MB-328 Xenograft Model:

Five-week-old athymic Nude-Foxn 1nu male mice (ENVIGO Labs, Nes Ziona, Israel) were subcutaneously inoculated on the dorsal left side with MDA-MB-328 (1×10⁶ cell) into nu/nu mice, 0.2 mL per mouse) and tumors allowed to establish over time. When tumor volume reached 100 mm³, Doxorubicin (Dox) and Doxorubizen (Chimera 9) were administered intraperitoneally (ip). Animals were removed from an experiment if either of the following conditions were fulfilled: reduction of 20% or more body weight; tumor volume>0.55 cm³. The tumor dimensions were measured using a Vernier caliper and the volume was calculated according to the equation: Volume (mm³) ¼ length×width×width×½ three times per week. The in vivo experiments were approved by the Animal Ethics Committee (Authorization number IL-179-06-19).

### Statistical Analysis:

All studies were performed at least 3 times unless otherwise stated. Data is presented as mean±standard deviation and the statistical significance of differences determined using unpaired two-tailed Student's t-tests (GraphPad Prism) with p-value<0.05 considered significant.

### Example 5

### In Vitro Cytotoxicity Screening of Conjugates

In order to explore the cytotoxic effect of the conjugates provided herein, the inventor focused on 10 hematological cell-lines: Granta, Rec-1, Mino and Jeko-1; mantle cell lymphoma (MCL) model. MUTZ5 and MHH-CALL4; B-cell Acute lymphocytic leukemia (B-cell ALL) model. DND41; T-ALL model. KG-1a and OCI-AML-3; Acute myeloid leukemia (AML) model and HEL; erythroleukemia model.

The cytotoxic effect of chimeras 1-9 was investigated by WST-1 proliferation assay. The WST-1 proliferation assay is a colorimetric assay that is used to measure cell proliferation. It is based on the cleavage of the tetrazolium salt WST-1 to formazan by cellular mitochondrial dehydrogenases. The amount of formazan produced is directly proportional to the number of living cells.

Chimera 1 and Chimera 9 (Doxorubizen) inhibited the proliferation rate in all the examined cell-lines. Chimeras 2, 4, 5, 6 and 7, decreased the proliferation rate in at least 3 cell-lines. Chimera 1, chimera 9 and chimera 7 inhibited cell proliferation at low doses (250 nM-2.5 μM) and therefore the following substrate pairs were investigated: chimera 1 vs SN-38, chimera 4 vs combretastin, chimera 9 vs doxorubicin, and chimera 7 vs riluzole. Among the four chimeras examined, chimera 1 and chimera 4 showed less than superior cytotoxic effect compared to the parental non-conjugated drug, whereas chimera 9 and chimera 7 showed reduced proliferation rate of numerous cell-lines treated with chimeras versus the corresponding non-conjugated drug controls.

Table 3 [resents the calculated IC₅0 values (μM) for each chimera in different hematological malignancies cell-lines. The proliferation rate was measured by WST-1 assay after 48 hours of treatment. The result shown for each concentration point represents the

TABLE 3

| | Cell | | | | | | | | | |
|---|---|---|---|---|---|---|---|---|---|---|
| Line | MHH- | | OCI- | | | | | | | |
| Chimera | CALL4 | Rec-1 | AML3 | MINO | MUTZ5 | KG-1a | JEKO-1 | HEL | GRANTA | DND41 |
| 1 | 1.2 | 0.59 | 12.3 | 0.63 | 3.02 | 1.04 | 0.36 | 0.55 | 8.42 | 1.99 |
| 2 | 24.04 | 14.16 | 17.10 | 5.7 | 47.51 | 46 | 22.81 | 1.17 | >50 | >50 |
| 3 | >50 | 23.72 | 10.22 | 30.33 | 34.77 | >50 | 28.63 | 43.1 | 42.1 | >50 |
| 4 | 21.56 | 0.13 | >50 | 0.69 | 39.81 | 4.72 | 13.8 | 1.63 | 41.49 | 27.59 |
| 5 | 32.38 | 0.13 | 22.71 | 29.74 | 44.62 | 27.7 | >50 | 2.72 | >50 | 30.88 |
| 6 | 16.6 | 20.47 | 8.76 | 6.88 | 30.97 | 39.2 | 39.52 | 13.5 | >50 | >50 |
| 7 | 5.25 | 17.99 | 14.21 | 7.8 | 1.81 | 23 | >50 | 2.37 | 43.04 | 2.37 |
| 8 | 35.96 | >50 | 12.72 | 13.3 | 10.27 | 19.1 | 7.12 | 1.97 | >50 | 20.92 |
| Doxorubizen | 1.14 | 0.52 | 1.24 | 0.48 | 0.52 | 0.4 | 0.85 | 0.97 | 2.59 | 0.64 |

mean ± standard error calculated from triplicates.

However, treatment with 250 nM of chimera 9 resulted in an exceptional cytotoxic effect. Therefore, it was decided to reduce chimera 9 and the parental drug (doxorubicin) doses to 50-250 nM range on the most four aggressive hematological malignancies cell-lines (OCI-AML3, KG-1a, Granta and Jeko-1). Moreover, chimera 9 was examined on an additional aggressive leukemia cell line (BV-173) and breast cancer cell-line model (MDA MB-231).

Treatment with chimera 9 in low doses (50 nM-250 nM) showed significant reduction in the proliferation rate of all tested cell-lines treated with chimeras versus the corresponding controls. As major drawbacks of doxorubicin therapy include primary resistance and secondary resistance acquired in the course of the treatment, the development of doxorubicin analogs with enhanced activity and low rate of the secondary resistance acquisition would be very beneficial.

### Example 6

### Evaluation of Doxorubizen on Aggressive Thyroid Cancer

Thyroid cancer accounts for 3-4% of cancers, it is one of the most common endocrine tumor, and its occurrence has risen in the last 10 years. Differentiated thyroid cancer (DTC) are papillary TC (PTC), follicular TC (FTC), and Hurthle cells TC. The overall survival (OS) (5- and 10-year) for DTC patients depends on several factors, such as the age at the diagnosis and cancer subtype, but in general PTC and FTC have slow progression rates and a 10-year-OS rate of 98%, and 92%, respectively. Conversely, anaplastic thyroid cancer (ATC) is rare and accounts for about 1% of thyroid cancer, but it is more aggressive leading to 15-40% of TC death. In patients with ATC the median survival is approximately 6 months, and while PTC and FTC have a slow progression rate, ATC is an undifferentiated tumor of the thyroid follicular epithelium, showing a much more aggressive behavior with respect to PTC and FTC. About 50% of ATC occurs after a past history of thyroid nodules, PTC or FTC. Owing to the ATC poor prognosis, multiple therapeutic strategies are usually attempted including debulking, external beam radiotherapy (EBRT), and chemotherapy (cisplatin or doxorubicin), reaching about 10 months of median survival. ATA guidelines indicate the efficacy of docetaxel or paclitaxel, doxorubicin, and also carboplatin, or cisplatin, in the treatments of ATC, but none of them have shown to improve OS. Current treatment strategies are not completely effective against ATC, and mortality is one of the most important challenges. In the last decades, thyroid continuous cell lines have been considered preclinical models for research purposes⁽¹⁷⁾. Continuous cell lines are used for convenience because they can proliferate indefinitely, are easy to handle, but actually they may have lost some of their thyroid specific characteristics compared to the beginning. The cells adapt to the in vitro growth conditions, and they are not able to maintain important features that are determinant in normal thyroid physiology and signaling pathways. Moreover, as reported by genetic analysis, conducted by short tandem repeats and SNP, several thyroid cell lines are misidentified or cross-contaminated with other cells, and they do not mimic closely the in vivo environment. For these reasons, the use of these cell lines for research has important limitations. In the last years, human primary thyroid cells have been studied as monolayer cultures, and their biological behavior have been investigated⁽¹⁷⁾. Primary human cells have some advantages than continuous cell lines in the study of the antineoplastic action of different drugs, because primary cells:
a) are more differentiated than continuous cell lines;
b) have phenotypic features that are quite similar to those of the primary tumor;
c) can be used for chemosensitivity tests in the single patient. Though primary cell cultures have a limited lifespan, they permit to investigate, not only cells, but also donors, as different factors (such as age, sex, medical history, race, etc.) can be considered during the evaluation of the experimental model, while continuous cell lines do not reproduce the true variability of a living tissue.

Chimera 9 (also referred to herein as Doxorubizen) was evaluated in two different concentrations (10 nM and 100 nM) on aggressive papillary thyroid cancer and with respect to the parent anticancer drug Doxorubicin. The results suggest that chimera 9 is able to reduce significantly the proliferation of thyroid cancer cells at 10 nM and 100 nM with respect to DMSO control and with respect to Doxorubicin, that had no effect on cell proliferation.

Human primary anaplastic thyroid cancer (ATC) cells were treated with chimera 9 (100 nM, 250 nM, and 500 nM), or Doxorubicin (100 nM, 250 nM, and 500 nM), or with vehicle, for 3, 5, and 7 days. Both chimera 9 and Doxorubicin reduced the vitality/proliferation of primary human ATC cells significantly in a dose-dependent and a time-dependent manner, with a higher effect exercised by chimera 9 vs. Doxorubicin.

### Example 7

### Doxorubizen Induce Mitochondrial Depolarization and Cell Death

Depolarization of mitochondrial membrane potential prevents calcium entry into the mitochondria. This is a critical step in the progression to cell death.

Chimera 9 was evaluated for its biological impact on mitochondrial function and cell death. To that end, the inventor treated the following cell lines OCI-AML3 (100 nM), KG-1a (100 nM), Granta (250 nM), Jeko-1 (50 nM) and BV-173 (50 nM) with doxorubicin or chimera 9.

The results clearly showed that treatment with chimera 9 significantly collapsed mitochondrial membrane potential and resulted in a substantial increase in the rate of apoptosis as compared to the parental drug in all tested cell-lines.

### Example 8

### Doxorubizen Induce Superior DNA Damage as Compared to the Parental Drug

BV-173 cells were exposed to 50 nM chimera 9 or the non-conjugated drug Doxorubicin, and DNA damage was analyzed after 3 hours by using dual staining of phospho-Histone H2A.X (Ser139) and phospho-ATM (Ser1981) antibodies.

The results show significant co-activation of pH2A.X and pATM upon treatment with chimera 9, indicating that chimera 9 induced substantial DNA damage and double-strand breaks in treated cells compared to the parental drug.

Chimera 9 was also evaluated for its effect on breast cancer model. For this evaluation, 12 nude female (6-8 weeks) mice (groups 1, 2, two mice per group, groups 3, 4, 5 three mice per group) were inoculated with triple negative breast cancer (TNBC) MDA 231 cells. Group 1 was used as the control for tumor growth. After 10 days, mice were perinatal (ip) administered with Doxorubicin (groups 2 and 4) and Chimera 9 (Doxorubizen) (groups 3 and 5) in the following regime: ip administration of 2.5 mg/kg Doxorubicin once per week (group 2) and three sequential days per week (group 4); ip administration of 3.5 mg/kg chimera 9 once per week (group 3) and three sequential days per week (group 5). Overall experiment carried out for 5 weeks and 4 days, when the tumor volumes for all the groups were monitored. Tumors were then resected and measured.

Group 1 mice were sacrificed at 18^{th} day and group 2 at 12^{th} day due to large tumor volumes and severe animal conditions. Group 4 mice died at 20^{th} day due to Doxorubicin over toxication. Group 5 presented full survival and were sacrificed at 31^{st} day due to accomplishment of the study. Group 3 showed no statistical significance compared to Group 2. Animals in this group where sacrificed due to tumor progression of each mouse.

Apparently, in TNBC MDA231 tumor bearing mice xenograft, chimera 9 was markedly superior upon doxorubicin in weight maintenance, survival and tumor growth suppression. All mice in Group 5 survived until the end of the experiment (32 days), didn't lose weight, and the tumor volume was even reduced pointing a reduced toxicity of chimera 9 vs doxorubicin.

### Experimental Conclusions

Topoisomerase II (TopII) poisons are compounds that increase levels of topoisomerase II-DNA cleavage complexes. Although the function of TopII poisons is not completely understood there is evidence that differences in structural specificity between intercalating and non-intercalating poisons like Doxorubicin and Etoposide respectively reflect on their mechanism of action. It is known that the difference between the two classifications of poisons rely on their biological activity and its role in the formation of the TopII-DNA covalent complexes. More specifically, this difference occurs between the chromophore framework and the base pairs of DNA. The inventor assume that this can be a reason why chimera 9 is superior to Doxorubicin, while Etoposide chimera 3 did not show prevalence over Etoposide. Such an assumption is supported by the results from a previous study with Amonafidazene, which is a structural hybrid of another DNA intercalator and Topo II poison Amonafide. Amonafidazene was superior in all biological parameters in vitro and in vivo over its parent substance Amonafide.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A compound, characterized by general Formula I: 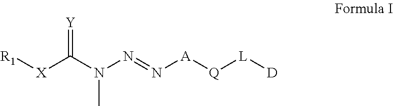
wherein:
D is a residue of a DNA intercalating agent;
L is a linking moiety that is not carbamate;
A is an aromatic or a heteroaromatic ring;
Q is selected from the group consisting of (CH₂)₁₋₁₀, C═O, C═NH, C═N-alkyl, C═N-aryl and C═S;
X and Y are each independently selected from the group consisting of O, S, N, NH, alkylene C₁₋₅, sulfone, and phosphonate; and
R₁ is selected from the group consisting of hydrogen, alkyl, alkoxyalkyl, alkoxy, aryl, heteroaryl, alkylaryl, o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, a tertiary alkyl amine, a quaternary ammonium choline, L-valine-L-citrulline-p-aminobenzyl alcohol (Val-Cit-PABA), L-valine-L-alanine-p-aminobenzyl alcohol (Val-Ala-PABA), or a protecting group.

2. The compound of claim 1, wherein D is an anticancer drug selected from the group consisting of Doxorubicin (Adriamycin), Daunorubicin (Daunomycin), Epirubicin (Ellence), Idarubicin (Idamycin), Annamycin, Camsirubicin, Berubicin, Daunomustine, Amonafide, Etoposide, Zorubicin, Carubicin, Losoxantrone, Pixantrone, Mitoxantrone, Pirarubicin, Sabarubicin, Amrubicin, and Aldoxorubicin.

3. The Compound of claim 1, wherein L is selected from the group consisting of amine, ether, thioether, amide, carbonate, lactone, lactam, carboxylate, ester, boroalkyl, boronate, sulphone, sulphate, phosphonate, phosphine, phosphite, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thiocarbamate, thioacetal, thioketal, phosphate ester, and the like.

4. The Compound of claim 3, wherein L is amine, ether, thiocarbamate, carbonate, or thioether.

5. The Compound of claim 1, wherein A is a substituted or unsubstituted aromatic or heteroaromatic ring selected from the group consisting of phenyl, naphthyl, anthryl, phenanthryl, biphenyl, furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, thiazole, bicyclo[4.4.0], bicyclo[4.3.0], indolizine, indole, isoindole, indazole, benzimidazole, benzthiazole, purine, quinoline, isoquinoline, cinnoline, phthalazine, quinazoline, quinoxaline, 1,8-naphthyridine, and pteridine.

6. The Compound of claim 5, wherein A is phenyl.

7. The Compound of claim 1, wherein X and Y are each O.

8. The compound of claim 1, each selected independently from the group consisting of Chimera 8, Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 24, Chimera 25, Chimera 26, Chimera 27, Chimera 28, Chimera 29, and Chimera 30.

9. A compound, characterized by general Formula II: 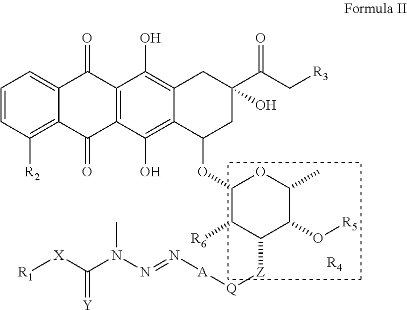
wherein:
R₁ is selected from the group consisting of hydrogen, alkyl, alkoxyalkyl, alkoxy, aryl, heteroaryl, alkylaryl, o-nitrobenzyl, benzhydryl, trityl, alkylsilyl, perillyl alcohol, ethyl(2-(thiomethyl)ethyl)sulfane, 2-((2-(propylthio)propan-2-yl)thio)ethan-1-ol, a tertiary alkyl amine, a quaternary ammonium choline, L-valine-L-citrulline-p-aminobenzyl alcohol (Val-Cit-PABA), L-valine-L-alanine-p-aminobenzyl alcohol (Val-Ala-PABA), or a protecting group;
each of R₂ and R₃ is independently selected from the group consisting of hydrogen, hydroxy, alkyl, alkoxyalkyl, alkoxy, aryl, heteroaryl, alkylaryl, ortho nitrobenzyl, benzhydryl, trityl, alkylsilyl, or a protecting group;
R₄ is selected from the group consisting of the (2R,4S,5S,6S), (2R,4S,5R,6S), (2R,4S,5S,6S) and (2R,4S,5S,6S) stereoisomer of 4-(λ¹-azaneyl)-2-methyl-6-(λ²-oxidaneyl)tetrahydro-2H-pyran-3-ol;
R₅ is selected from the group consisting of hydrogen, alkyl, aryl, heteroaryl and benzyl;
R₆ is selected from the group consisting of hydrogen, F, Cl, Br and I;
Z is selected from the group consisting of O, S, NH and N-alkyl;
Q is selected from the group consisting of (CH₂)₁₋₁₀, C═O, C═NH, C═N-alkyl, C═N-aryl and C═S;
A is an aromatic or a heteroaromatic ring; and
X and Y are each independently selected from the group consisting of O, S, N, NH, alkylene (C₁₋₅), sulfone, and phosphonate.

10. The Compound of claim 9, wherein R₅ and R₆ is each independently hydrogen.

11. The Compound of claim 9, wherein Z is NH.

12. The Compound of claim 9, wherein Q is CH₂.

13. The Compound of claim 9, wherein A is phenyl.

14. The Compound of claim 9, wherein X and Y are each O.

15. The compound of claim 9, each selected independently from the group consisting of Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 24, Chimera 25, Chimera 26, Chimera 27, Chimera 28, Chimera 29, and Chimera 30.

16. A compound selected independently from the group consisting of Chimera 8, Chimera 9, Chimera 10, Chimera 11, Chimera 12, Chimera 13, Chimera 14, Chimera 15, Chimera 16, Chimera 17, Chimera 18, Chimera 19, Chimera 20, Chimera 21, Chimera 22, Chimera 24, Chimera 25, Chimera 26, Chimera 27, Chimera 28, Chimera 29, and Chimera 30.

17. (canceled)

18. A pharmaceutical composition comprising a therapeutically effective amount of the compound of claim 1, and/or any pharmaceutically acceptable salt, a prodrug, an ester, a solvate, and/or hydrate thereof, and a pharmaceutically acceptable carrier, diluent or vehicle.

19-20. (canceled)

21. A method for treating cancer, comprising administering to a subject in need thereof a pharmaceutically effective amount of the compound of claim 1 and/or any pharmaceutically acceptable salt, a prodrug, an ester, a solvate, and/or hydrate thereof.
